(19) United States
(12) Patent Application Publication
(10) Pub. No.: US 2007/0009603 A1
(43) Pub. Date: Jan. 11, 2007

(54) Compositions comprising fenofibrate and atorvastatin

(76) Inventors: Per Holm, Vanlose (DK); Tomas Norling, Lyngby (DK)

Correspondence Address: CHERYL H AGRIS PHD, PO BOX 806, PELHAM, NY, 10803, US

(21) Appl. No.: 10/988,917

(22) Filed: Nov. 15, 2004

Related U.S. Application Data
(63) Continuation-in-part of application No. PCT/DK04/00668, filed on Oct. 1, 2004.

(30) Foreign Application Priority Data
Oct. 10, 2003 (DK) PA 2003 01503
Mar. 23, 2004 (DK) PA 2004 00464

(51) Int. Cl. A61K 31/401; A61K 31/192; A61K 9/14

(52) U.S. Cl. 424/489; 514/423; 514/571

(57) ABSTRACT

The present invention relates to pharmaceutical compositions in particulate form or in solid dosage forms comprising a combination of fenofibrate and the HMG CoA reductase inhibitor atorvastatin or a pharmaceutically active salt thereof, which upon oral administration provides a relative AUC₀₋₂₄ value (AUC_{fibric acid}/AUCₐₜₒᵣᵥₐₛₜₐₜᵢₙ) of between about 250 and about 10,000. The solid compositions are manufactured without any need of addition of water or aqueous medium and comprise at least 80% of the active substances fenofibrate and atorvastatin in dissolved form, or, optionally, atorvastatin in micronized form, in order to ensure suitable bioavailability.

### CROSS REFERENCE TO RELATED APPLICATIONS

This application claims priority under 35 USC 119 (a)-(d) from Danish application no. PA 2003 01503, filed Oct. 10, 2003, Danish Patent Application No. PA 2004 00464, filed Mar. 23, 2004 and is a continuation-in-part application of PCT/DK2004/000668, filed Oct. 1, 2004, the contents of each of which are incorporated herein by reference.

### FIELD OF THE INVENTION

The present invention relates to compositions, particularly, pharmaceutical compositions in particulate form such as granulate or in solid dosage forms comprising a combination of a fibrate, namely fenofibrate, and a statin (also known as a HMG CoA reductase inhibitor), namely atorvastatin, in optimized relative amounts providing effective AUC₀₋₂₄ when administered orally to mammals. Further, the invention relates to methods for making the compositions in particulate form, i.e. as particles, and in solid dosage forms.

### BACKGROUND OF THE INVENTION

Fibrates are drug substances that generally are poorly and variably absorbed after oral administration. Normally they are prescribed to be taken with food in order to increase the bioavailability. There has been a number of improvements in dosage form of the currently most used fibrate, fenofibrate, in an effort to increase the bioavailability of the drug and hence its efficacy. Furthermore, clinical guidelines indicate that not only fibrate therapy but also a combination therapy with e.g. fenofibrate and a statin should be the most effective means of cholesterol and lipid management. In fact, treatment with fenofibrate is often prescribed together with a statin as clinicians seem to prefer the use of e.g. fenofibrate due to its triglyceride-lowering and HDL-C increasing effects while a statin is used for its positive effects on lowering LDL-C and raising HDL-C. However, at present, such a combination therapy can only be achieved by the use of two separate products, i.e. the patient needs to take e.g. one fenofibrate tablet together with another tablet or capsule containing a statin.

Fenofibrate is chemically named 2-[4-(4-chlorobenzoyl]-2-methyl-propanoic acid, 1-methylethyl ester and has the following structural formula:

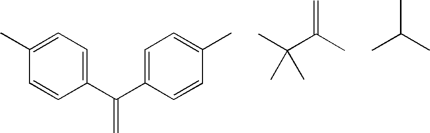

Fenofibrate is a white solid. The compound is insoluble in water. The melting point is 79-82° C. Fenofibrate is metabolised to the active substance fenofibric acid. Fenofibric acid has an elimination half-life of about 20 hours. Measurement of the detected amount of fenofibric acid in the blood of a patient can reflect the efficacy of fenofibrate uptake. Fenofibric acid produces reductions in total cholesterol (total-C), LDL-C, apo-lipoprotein B, total triglycerides, and triglyceride rich lipoprotein (VLDL) in treated patients. In addition, treatment with fenofibrate results in increases in high density lipoprotein (HDL) and apolipoprotein apoAI and apo AII. Fenofibrate acts as a potent lipid regulating agent offering unique and clinical advantages over existing products in the fibrate family of drug substances. Fenofibrate produces substantial reduction in plasma triglyceride levels in hypertriglyceridemic patients and in plasma cholesterol and LDL-C in hypercholesterolemic and mixed dyslipidemic patients.

Fenofibrate also reduces serum uric acid levels in hyperuricemic and normal subjects by increasing the urinary excretion of uric acid.

Clinical studies have demonstrated that elevated levels of total cholesterol, low density lipoprotein cholesterol (LDL-C), and apo-lipoprotein B (apo B) are associated with human atherosclerosis. Decreased levels of high density lipoprotein cholesterol (HDL-C) and its transport complex, apolipoprotein A (apo AI and apo AII) are associated with the development of atherosclerosis.

Fenofibrate is also effective in the treatment of Diabetes Type II and metabolic syndrome.

Fenofibrate is also indicated as adjunctive therapy to diet for treatment of adult patients with hypertriglyceridemia (Fredrickson Types IV and V hyperlipedemia). Improving glycemic control in diabetic patients showing fasting chylomicronemia will usually reduce fasting triglycerides and eliminate chylomicronemia and thereby obviating the need for pharmacologic intervention.

Fibrates are drug substances known to be are poorly and variably absorbed after oral administration. Normally they are prescribed to be taken with food in order to increase the bioavailability.

As mentioned above, there has been an interest in obtaining improved compositions of fenofibrate and, accordingly, a number of publications relating to such compositions have emerged recently (see e.g. WO 04/041250). Although such compositions may lead to an improved fibrate therapy they do not meet the need for providing a composition containing a combination of a fibrate and a statin that is stable with respect to storage stability and at the same time leads to a suitable bioavailability of both active substances.

Atorvastatin calcium is (R—(R*, R*)]-2-(4-fluorophenyl)-β,δ-dihydroxy-5-(1-methylethyl)-3-phenyl-4-[(phenylamino)carbonyl]-1H-pyrrole-1-heptanoic acid, calcium salt (2:1) trihydrate. The empirical formula of atorvastatin calcium is (C₃₃H₃₄ FN₂O₅)2Ca.3H₂O and its molecular weight is 1209.42.

Atorvastatin calcium is a white to off-white crystalline powder that is insoluble in aqueous solutions of pH 4 and below. Atorvastatin calcium is very slightly soluble in distilled water, pH 7.4 phosphate buffer, and acetonitrile, slightly soluble in ethanol, and freely soluble in methanol. The calcium salt has the following structural formula:

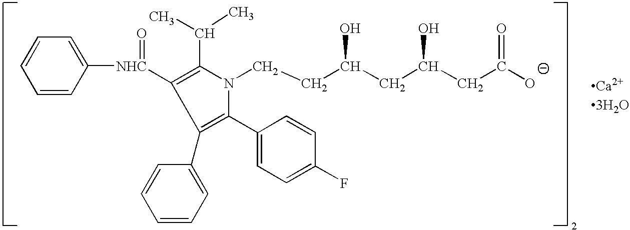

Lipitor™ tablets (from Pfizer Inc.) for oral administration contain 10, 20, 40 or 80 mg atorvastatin and the following inactive ingredients: calcium carbonate, USP; candelilla wax, FCC; croscarmellose sodium, NF; hydroxypropyl cellulose, NF; lactose monohydrate, NF; magnesium stearate, NF; microcrystalline cellulose, NF; Opadry White YS-1-7040 (hydroxypropyl-methylcellulose, polyethylene glycol, talc, titanium dioxide); polysorbate 80, NF; simethicone emulsion.

Atorvastatin is a synthetic lipid-lowering agent. Atorvastatin is an inhibitor of 3-hydroxy-3-methylglutaryl-coenzyme A (HMG-CoA) reductase. This enzyme catalyzes the conversion of HMG-CoA to mevalonate, an early and rate-limiting step in cholesterol biosynthesis.

Atorvastatin is a selective, competitive inhibitor of HMG-CoA reductase, the rate-limiting enzyme that converts 3-hydroxy-3-methylglutaryl-coenzyme A to mevalonate, a precursor of sterols, including cholesterol. Cholesterol and triglycerides circulate in the bloodstream as part of lipoprotein complexes. With ultracentrifugation, these complexes separate into HDL (high-density lipoprotein), IDL (intermediate-density lipoprotein), LDL (low-density lipoprotein), and VLDL (very-low-density lipoprotein) fractions. Triglycerides (TG) and cholesterol in the liver are incorporated into VLDL and released into the plasma for delivery to peripheral tissues. LDL is formed from VLDL and is catabolized primarily through the high-affinity LDL receptor. Clinical and pathologic studies show that elevated plasma levels of total cholesterol (total-C), LDL-cholesterol (LDL-C), and apolipoprotein B (apo B) promote human atherosclerosis and are risk factors for developing cardiovascular disease, while increased levels of HDL-C are associated with a decreased cardiovascular risk.

Atorvastatin is rapidly absorbed after oral administration; maximum plasma concentrations occur within 1 to 2 hours. Extent of absorption increases in proportion to atorvastatin dose. The absolute bioavailability of atorvastatin (parent drug) is approximately 14% and the systemic availability of HMG-CoA reductase inhibitory activity is approximately 30%. The low systemic availability is attributed to presystemic clearance in gastrointestinal mucosa and/or hepatic first-pass metabolism. Although food decreases the rate and extent of drug absorption by approximately 25% and 9%, respectively, as assessed by cₘₐₓ and AUC, LDL-C reduction is said to be similar whether atorvastatin is given with or without food. Plasma atorvastatin concentrations are lower (approximately 30% for cₘₐₓ and AUC) following evening drug administration compared with morning. However, LDL-C reduction is said to be the same regardless of the time of day of drug administration

In general, it is known that the absorption and bioavailability of a therapeutically active substance can be affected by a variety of factors when administered orally. Such factors include the presence of food in the gastrointestinal tract and, in general, the gastric residence time of a drug substance is significantly longer in the presence of food than in the fasted state. If the bioavailability of a drug substance is affected beyond a certain point due to the presence of food in the gastrointestinal tract, the drug substance is said to exhibit a food effect. Food effects are important because there is a risk associated with administering the drug substance to a patient who has eaten recently. The risk derives from the potential that absorption into the bloodstream may be adversely affected to the point that the patient risks insufficient absorption to remedy the condition for which the drug was administered. In the case of e.g. fenofibrate the situation is different in that food increases the uptake. Thus, lack of intake of food simultaneously with the drug substances may lead to insufficient absorption. The extent of absorption of a commercially available product TRICOR® containing fenofibrate (from Abbott) is increased by approximately 35% under fed as compared to fasting conditions.

WO 03/013608 describes compositions containing a fibrate and a statin. However, due to the manufacturing process (the active substances are melted together, filled into gelatin capsules and allowed to cool) only capsules can be prepared. Furthermore, from a pharmaceutical point of view the manufacturing process seems to be difficult to up-scale taken into consideration the regulatory requirements with respect to e.g., mass variation, variation in drug content etc. Although the composition may appear as a solid composition, there seems to be no flexibility in the formulation principle to provide other types of dosage forms than capsules.

Accordingly, there is a need for developing a pharmaceutical composition that in a single formulation contains a fibrate and a statin as active substances, which composition is stable and provides suitable biopharmaceutical properties to the active substances (e.g. suitable bioavailability, less dependency on food intake etc), and which composition easily can be manufactured in large scale. Furthermore, there is a need for developing formulations containing a fibrate and a statin, which formulations can be further processed into pharmaceutical dosage forms with a high degree of flexibility of choosing the particular kind of dosage form. Within the pharmaceutical field such flexibility can be obtained when the formulation is in the form of a solid product such as powder or particles. Accordingly, the present invention provides such a particulate material suitable for further processing into e.g. tablets.

In addition, there is still a need for a composition that has a suitable bioavailability, that can substantially reduce or overcome the differential between the bioavailability of the drug in patients who are fasted versus the bioavailability of the drug (in particular relevant for fenofibrate) in patients who are fed, and/or than can substantially reduce or overcome the intra- and/or inter-individual variations observed with the current treatment. Furthermore, there is also a need for a composition that enables reduction in observed side effects.

### SUMMARY OF THE INVENTION

The inventors have now provided a drug combination composition comprising two active substances, fenofibrate and atorvastatin, or a pharmaceutically active salt thereof such as atorvastatin calcium, which after oral administration to a mammal provides a relative AUC₀₋₂₄ value (AUC_{fibric acid}/AUCₐₜₒᵣᵥₐₛₜₐₜᵢₙ) of between about 250 and about 10,000.

Further, the inventors have found that the bioavailabilty of the active substances can be significantly enhanced by dissolving the compounds in a suitable vehicle and using the resulting composition for preparing a solid dosage form, i.e. a dosage form excluding material in liquid form. Fenofibrate is known to be insoluble in water and atorvastatin is sparingly soluble in water, but the present invention provides pharmaceutical compositions and formulations exhibiting immediate release profiles which are contemplated having significantly increased in vivo bioavailability in patients in need thereof. Especially, the inventors have succeeded in preparing a solid dosage form, such as a tablet, wherein at least 80% of the active substances (i.e., fenofibrate and atorvastatin or, alternatively, only the fenofibrate) are present in the composition in dissolved form, which ensures suitable bioavailability of both active ingredients upon oral administration. The advantages of a solid and stable dosage form useful for oral administration are well-known.

The compositions, i.e., the particulate material and the solid dosage forms, are manufactured without any need of addition of water or an aqueous medium. As a result, the compositions of the invention have a very low content of moisture, i.e. less than about 2.5% w/w water, or less than about 2% w/w water, or less than about 1% w/w water are obtained, thereby ensuring suitable storage stability, since both fibrates and statins are degradable by water.

Thus, the present invention provides pharmaceutical compositions in the form of particulate material and solid dosage forms useful for treatment of conditions that respond to fibrate and statin treatment.

Accordingly, in a first aspect, the present invention provides a particulate material comprising as an active substance fenofibrate and atorvastatin or a pharmaceutically active salt thereof, which provides a relative AUC₀₋₂₄ value (AUC_{fibric acid}/AUCₐₜₒᵣᵥₐₛₜₐₜᵢₙ) of between about 250 and about 10,000 when administered orally to a mammal, the AUC values being determined from steady state plasma concentrations of fibric acid and atorvastatin, respectively. Especially, about 80% or more of the active substances are dissolved in a vehicle, which is hydrophobic, hydrophilic or water-miscible.

In a second aspect, the invention relates to a solid oral dosage form comprising the particulate material. Useful solid dosage forms are in the form of tablets, beads, capsules, grains, pills, granulate, granules, powder, pellets, sachets or troches.

In yet another aspect, the invention relates to a method of manufacturing the particulate material and the solid oral dosage form of the invention.

Further aspects of the invention are evident from the following description. Comparison in vivo tests in dogs have shown, cf. the examples herein, that solid dosage forms and compositions of the invention exhibit significantly enhanced bioavailability of fenofibrate compared to commercially available solid dosage forms containing the same active ingredient, i.e. to TRICOR® tablets and LIPANTHYL® capsules (both from Abbott Laboratories, Illinois).

Further, it is strongly believed that the present invention provides solid dosage forms and/or compositions of fibrate capable of significantly reducing the intra- and/or inter-individual variation normally observed after oral administration. Furthermore, compositions and/or dosage forms according to the invention provide for a significant reduced food effect, i.e. the absorption is relatively independent on whether the patient takes the composition or dosage form together with or without any meal. It is contemplated that a modified release of the fibrate may reduce the number of gastro-intestinal related side effects. Furthermore, it is contemplated that a significantly larger amount of the fibrate is absorbed and, accordingly, an equally less amount is excreted unchanged via feces.

As mentioned above, the present invention fulfills the need for pharmaceutical compositions containing a combination of fenofibrate and atorvastatin or a pharmaceutically acceptable salt thereof for oral use that lead to an improved treatment of conditions requiring lipid management (e.g., atherosclerosis, coronary heart diseases, diabetes management, obesity, overweight, metabolic syndrome etc.)

Furthermore, it is contemplated that the invention provides improved bioavailability, especially of the fibrate component, but as described below in some cases also for the statin component. A fibrate like fenofibrate has a very poor solubility in water, which property is regarded as one of the major reasons for the poor bioavailability of fenofibrate. Accordingly, it is advantageous to provide a composition in which the fenofibrate is mainly in dissolved form. The same applies to atorvastatin that also has poor solubility in water.

Improved bioavailability results in improved treatment. However, it may also be possible to obtain the same therapeutic response with a decreased dose and/or a less frequent administration and less variability in plasma levels and no food restrictions. Another way of obtaining an improved treatment of conditions where e.g., fenofibrate is indicated is by balancing the release of fenofibrate to the gastrointestinal tract in such a manner that an enhanced plasma concentration of fenofibrate is obtained initially or delayed with respect to the time of administration. A further therapeutic improvement is development of modified or delayed release compositions containing one or more fibrates.

Especially, the invention provides a solid composition in particulate form that can be further processed into solid dosage form (e.g., tablets etc.). Such a composition contains the active drug substances, i.e., fenofibrate and atorvastatin, mainly in dissolved form, but at the same time the composition is physically in particular form, i.e. in form of solid particles, that can be further processed into a solid dosage form like e.g. tablets. Accordingly, the particulate material containing the active substances mainly in dissolved form exhibits suitable properties such as, e.g., flowability (free-flowing), adherence (which should be avoided), compressibility etc.

### DESCRIPTION OF THE INVENTION

Definitions

As used herein, the term “active substance”, “active pharmaceutical substance”, “active ingredient” or “active pharmaceutical ingredient” means any component that is intended to furnish pharmacological activity or other direct effect in the diagnosis, cure, mitigation, treatment, or prevention of disease, or to affect the structure or any function of the body of man or other animals. The term includes those components that may undergo chemical change in the manufacture of the drug product and are present in the drug product in a modified form intended to furnish the specified activity or effect.

In the present context, the term “hydrophilic” describes that something ‘likes water’, i.e. a hydrophilic molecule or portion of a molecule is one that typically is electrically polarized and capable of forming hydrogen bonds with water molecules, enabling it dissolve more readily in water than in oil or other “non-polar” solvents.

In the present context, the term “amphiphilic” describes a molecule (as a surfactant) having a polar water-soluble group attached to a water-insoluble hydrocarbon chain. Thus, one end of the molecule is hydrophilic (polar) and the other is hydrophobic (non-polar).

In the present context, the term “hydrophobic” denotes a compound tending to be electrically neutral and non-polar, and thus preferring other neutral and nonpolar solvents or molecular environments.

As used herein, the term “water-miscible” denotes a compound being fully or partly miscible with water. For example, certain polar lipids are partly water-miscible.

As used herein, the term “vehicle” means any solvent or carrier in a pharmaceutical product that has no pharmacological role. For example, water is the vehicle for xylocaine and propylene glycol is the vehicle for many antibiotics.

In the present context, the term “solid dispersion” denotes a drug or active ingredient or substance dispersed on a particulate level in an inert vehicle, carrier, diluent or matrix in the solid state, i.e. usually a fine particulate dispersion.

In the present context, the term “solid solution” denotes a drug or active ingredient or substance dissolved on a molecular level in an inert vehicle, carrier, diluent or matrix in the solid state.

As used herein, the term “analog” means a chemical compound that is structurally similar to another.

The term “drug” means a compound intended for use in diagnosis, cure, mitigation, treatment, or prevention of disease in man or other animals.

In this context, the term “dosage form” means the form in which the drug is delivered to the patient. This could be parenteral, topical, tablet, oral (liquid or dissolved powder), suppository, inhalation, transdermal, etc.

As used herein, the term “bioavailability” denotes the degree means to which a drug or other substance becomes available to the target tissue after administration. In the present context, the term “suitable bioavailability” is intended to mean that administration of a composition according to the invention will result in a bioavailability that is improved compared to the bioavailability obtained after administration of the active substance(s) in a plain tablet; or the bioavailability is at least the same or improved compared to the bioavailability obtained after administration of a commercially available product containing the same active substance(s) in the same amounts. In particular it is desired to obtain quicker and larger and/or more complete uptake of the active compound, and thereby provide for a reduction of the administered dosages or for a reduction in the number of daily administrations. Further, pharmaceutical compositions of the invention may also reduce or negate the need for food to be takes simultaneously with the dosage form (in particular relevant for one or the active substances contained in a composition of the invention, namely fenofibrate) thereby allowing patients more freedom on when the drug is taken.

As used herein, the term “bioequivalency” denotes a scientific basis on which generic and brand name drugs are compared with one another. For example, drugs are bioequivalent if they enter circulation at the same rate when given in similar doses under similar conditions. Parameters often used in bioequivalence studies are tₘₐₓ, cₘₐₓ, AUC_{0-infinity}, AUC₀₋ₜ. Other relevant parameters may be W₅₀, W₇₅ and/or MRT. Accordingly, at least one of these parameters may be applied when determining whether bioequivalence is present. Furthermore, in the present context, two compositions are regarded as bioequivalent if the value of the parameter used is within about 80-125% of that of PROGRAF® or a similar commercially available tacrolimus-containing product used in the test.

In the present context “tₘₐₓ” denotes the time to reach the maximal plasma concentration (cₘₐₓ) after administration; AUC_{0-infinity} or AUC denotes the area under the plasma concentration versus time curve from time 0 to infinity; AUC₀₋ₜ denotes the area under the plasma concentration versus time curve from time 0 to time t, especially, AUC₀₋₂₄ is the area under the plasma concentration versus time curve from time 0 to time 24 hr at steady state conditions; W₅₀ denotes the time where the plasma concentration is 50% or more of Cₘₐₓ; W₇₅ denotes the time where the plasma concentration is 75% or more of Cₘₐₓ; and MRT denotes mean residence time for a fibrate such as fenofibrate (and/or an analog thereof.

In this context, the term “medicine” means a compound used to treat disease, injury or pain. Medicine is designated “prophylactic,” i.e. the art of preserving health, and “therapeutic”, i.e. the art of restoring health.

In the present context, the terms “controlled release” and “modified release” are intended to be equivalent terms covering any type of release of tacrolimus from a composition of the invention that is appropriate to obtain a specific therapeutic or prophylactic response after administration to a subject. A person skilled in the art knows how controlled release/modified release differs from the release of plain tablets or capsules. The terms “release in a controlled manner” or “release in a modified manner” have the same meaning as stated above. The terms include slow release (that results in a lower Cₘₐₓ and later tₘₐₓ, but the half-life remains unchanged), extended release (that results in a lower Cₘₐₓ, later tₘₐₓ, but apparent half-life is longer); delayed release (that result in an unchanged Cₘₐₓ, but lag time and, accordingly, tₘₐₓ is delayed, and the half-life remains unchanged) as well as pulsatile release, burst release, sustained release, prolonged release, chrono-optimized release, fast release (to obtain an enhanced onset of action) etc. Included in the terms is also e.g. utilization of specific conditions within the body e.g., different enzymes or pH changes in order to control the release of the drug substance.

In this context, the term “erosion” or “eroding” means a gradual breakdown of the surface of a material or structure, for example of a tablet or the coating of a tablet.

The Active Drug Substances

A first drug or active substance of the dosage forms and pharmaceutical compositions of this invention is fenofibrate as described above or an analog thereof. It should be understood that this invention includes dosage forms and compositions comprising a mixture of two, three or even four different fibrates and/or fibric acids. Examples of other useful fibrates are bezafibrate, ciprofibrate, clinofibrate, clofibrate, etofylline, clofibrate, fenofibrate, gemfibrozil, pirifibrate, simfibrate and tocofibrate; particularly useful are gemfibrozil, fenofibrate, bezafibrate, clofibrate, ciprofibrate and active metabolites and analogues thereof including any relevant fibric acid such as fenofibric acid.

A second drug or active substance of the dosage forms and pharmaceutical compositions of this invention is atorvastatin as described above or a pharmaceutically acceptable salt thereof such as the sodium salt.

The first and second active substance, i.e. fenofibrate and atorvastatin, is present in the particulate material or the solid dosage form of the invention in a-relative amount so as to provide a relative AUC₀₋₂₄ value (AUC_{fibric acid}/AUCₐₜₒᵣᵥₐₛₜₐₜᵢₙ) of between about 250 and about 10,000 when administered orally to a mammal, the AUC values being determined from measurements of steady state plasma concentrations of fibric acid and atorvastatin, respectively. This results in an improved treatment of the patients, since it is believed that fenofibrate in combination with atorvastatin has an added affect; it has been shown that use of the combination results in TG and LDL levels being more decreased while HDL level is increased. More specifically, there is provided a relative AUC₀₋₂₄value of at least about 250, or at least about 500, or at least about 980, or at least about 2000; or a relative AUC₀₋₂₄value of less than about 10,000, or less than about 5100, or less than about 4000, or less than about 2100.

The fenofibrate of the particulate material or the solid dosage form of this invention provides, after oral administration, an AUC₀₋₂₄value of fibric acid (arithmetic mean) of at least 28,000 h·ng/mL, or at least of about 40,000 h·ng/mL, or at least of about 79,000 h·ng/mL, or at least of about 118,000 h·ng/mL.

In the particulate material or the solid dosage form of this invention at least about 80% w/w of the total amount of active substances or at least of fenofibrate is dissolved in vehicle selected from the group consisting of a hydrophobic, a hydrophilic and a water-miscible vehicle.

Normally, at least about 85% w/w, at least about 90% w/w, at least about 95% w/w or at least about 98% w/w, or at least about 99% w/w, or at least about 99.5% w/w of the total amount of active substances or at least of fenofibrate is dissolved in the vehicle.

If those embodiments where 100% of the active substances are dissolved in the vehicle, the active substances are present in the form of a solid solution in the particulate composition. The presence of a solid solution can be tested by a DSC test mentioned herein. However, some crystallization of the active substances from solid solutions may be expected during storage. Accordingly, the present invention includes particulate material wherein the active substances are present in the form of a solid solution, but it is within the scope of the present invention that the active substances may precipitate upon storage.

In another embodiment of the invention, at least about 80% w/w of fenofibrate is dissolved in the vehicle, which is further processed into particulate form as described herein. The solid particles, for examples granulate, comprising the dissolved fenofibrate is then mixed or blended with micronized atorvastatin, and the resulting composition is optionally subjected to conventional methods for preparing solid dosage forms, especially tablets.

As mentioned above, sufficient flowability is required of the particulate material according to the invention in order to obtain a suitable flexibility so that different dosage forms can be obtained. Accordingly, a particulate material according to the invention has a suitable flowability as determined according to the method described in the European Pharmacopoeia (Ph.Eur.) measuring the flow rate of the composition out of a funnel with a nozzle diameter of 10.0 mm. In a preferred embodiment, the particulate material is free-flowing.

In a specific embodiment, the concentration of fenofibrate in the vehicle is at least about 10% w/w, based on the total weight of the fibrate, the statin and the vehicle. In particular, the concentration of fenofibrate in the vehicle is at least about 15% w/w, or at least about 16% w/w, or at least about 17% w/w, or at least about 20% w/w, preferably at least 25% w/w, more preferably at least about 30% w/w, especially at least about 35% w/w, based on the total weight of the fibrate, the statin and the vehicle.

The concentration of atorvastatin in the vehicle of a particulate material or solid dosage form according to the invention is at least about 1% w/w, based on the total weight of the fibrate, the statin and the vehicle. More specifically, the concentration of statin in the vehicle is at least about 1.5% w/w, or at least about 2.5% w/w, or at least about 5% w/w, or at least about 7.5% w/w or at least about 10% w/w, based on the total weight of the fibrate, the statin and the vehicle.

The present invention provides particulate material and solid dosage forms for improved treatment of conditions that respond to fenofibrate and atorvastatin treatment.

Bioavailability

As described above, there remains a need for new pharmaceutical compositions comprising fenofibrate and atorvastatin exhibiting suitable bioavailability of the active substances and/or reduced or eliminated food effect. Administration of a composition according to the invention will result in a bioavailability that is improved compared to the bioavailability obtained after administration of the active substance(s) in a plain tablet; or the bioavailability is at least the same or improved compared to the bioavailability obtained after administration of a commercially available product containing the same active substance(s) in the same amounts. In particular it is desired to obtain quicker and larger and/or more complete uptake of the active compound, and thereby provide for a reduction of the administered dosages or for a reduction in the number of daily administrations. Further, pharmaceutical compositions of the invention may also reduce or negate the need for food to be takes simultaneously with the dosage form (in particular relevant for one or the active substances contained in a composition of the invention, namely fenofibrate) thereby allowing patients more freedom on when the drug is taken. Also, improved or enhanced bioavailability will lead to an improved treatment because it will be possible to obtain the same therapeutic response with a decreased dose and/or a less frequent administration and less variability in plasma levels and no food restrictions. Another way of obtaining an improved treatment of conditions where e.g., fenofibrate is indicated is by balancing the release of fenofibrate to the gastro-intestinal tract in such a manner that an enhanced plasma concentration of fenofibrate is obtained initially or delayed with respect to the time of administration, i.e. by applying modified or delayed release compositions containing one or more fibrates.

In one embodiment, the invention relates to a pharmaceutical composition in particulate form or solid dosage form comprising fenofibrate and atorvastatin, wherein the composition upon oral administration to a mammal in need thereof exhibits an AUC/AUC_{Control} value of at least about 1.0, the AUC_{Control} being determined using a commercially available product containing fenofibrate, and the AUC values being determined under similar conditions.

No absolute bioavailability data based on an injectable composition are available e.g., for fenofibrate (most likely due to solubility problems in aqueous media). The commercially available compositions containing fenofibrate include surface-active agents and/or e.g., a lipophilic medium. The surface-active agents may impart improved bioavailability and therefore, the bioavailability of such a composition may be sufficient already. However, there is still a need for developing a flexible formulation technique that enables preparation of a variety of dosage forms. Accordingly, the requirement to such improved and/or more flexible compositions may be to obtain the same or better bioavailability than already seen from the commercially available products.

Accordingly, in further embodiments of the invention, the AUC/AUC_{Control} value obtained by administering the solid dosage form or pharmaceutical composition of the invention is at least about 1.1 such as, e.g., at least about 1.2, at least about 1.3, at least about 1.4, at least about 1.5, about 1.75 or more, about 1.8 or more, about 1.9 or more, about 2.0 or more, about 2.5 or more, about 2.75 or more, about 3.0 or more, about 3.25 or more, about 3.5 or more, about 3.75 or more, about 4.0 or more, about 4.25 or more, about 4.5 or more, about 4.75 or more or about 5.0 or more, the AUC values being determined under similar conditions.

Likewise, the cₘₐₓ value obtained by administering the solid dosage form or pharmaceutical composition of the invention relative to the cₘₐₓ value of commercially available TRICOR® tablets is at least about 1.1, or at least about 1.2, or at least about 1.3, or at least about 1.4, or at least about 1.5, or at least about 1.6 or more, or at least about 2.0, or at least about 2.5, or at least about 3.0, the cₘₐₓ values being determined under similar conditions.

Another object of the invention is to reduce or eliminate the food effect. Thus, in another aspect, the invention relates to a pharmaceutical composition in particulate form or solid dosage form comprising one or more fibrates, especially fenofibrate, wherein the composition or solid dosage form upon oral administration to a mammal in need thereof does not exhibit a significant adverse food effect as evidenced by a value of (AUC_{fed}/AUC_{fasted}) of at least about 0.85 with a lower 90% confidence limit of at least 0.75. In a specific embodiment, the pharmaceutical composition or solid dosage form of the invention has a value of (AUC_{fed}/AUC_{fasted}) that is about 0.9 or more such as, e.g., about 0.95 or more, about 0.97 or more or about 1 or more.

In other words, the difference between a bioequivalence parameter measured after oral administration to a mammal with and without food, respectively, is less than about 25% such as, e.g., less than about 20%, less than about 15%, less than about 10% or less than about 5%.

In another aspect, the invention relates to a pharmaceutical composition in particulate form or solid dosage form comprising fenofibrate, wherein the composition upon oral administration to a mammal in need thereof is essentially bioequivalent with a commercially available product containing fenofibrate when administered in the same or lower dose as the commercially available product containing fenofibrate.

In specific embodiments thereof, the dose is at the most about 98% w/w such as, e.g., at the most about 95% w/w, at the most about 90% w/w, at the most about 85% w/w, at the most about 80% w/w, at the most about 75% w/w, at the most about 70% w/w, at the most about 65% w/w, at the most about 60% w/w, at the most about 55% w/w or at the most about 50% w/w of the dose of fenofibrate administered in the form of a commercially available product containing fenofibrate.

Normally, the bioequivalence is determined by means of at least one of the following parameters: tₘₐₓ (time to reach maximal plasma concentration), cₘₐₓ(maximal plasma concentration), AUC₀₋ₜ (area under the curve from time 0 to time t such as the time 24 h), AUC_{0-infinity} (area under the curve from time 0 to time infinity), W₅₀ (time period where the plasma concentration is 50% or more of cₘₐₓ), W₇₅ ((time period where the plasma concentration is 75% or more of cₘₐₓ) and/or MRT (mean residence time).

A major problem with treatment with fenofibrate is the large intra- or inter-individual variation. Thus, in a further aspect the invention relates to a pharmaceutical composition in particulate form comprising fenofibrate, wherein the composition upon oral administration to a mammal in need thereof reduces inter- and/or intra-individual variations compared to those of a commercially available product containing fenofibrate under the same conditions and in a dose that provides an equivalent therapeutic effect.

In the comparison tests mentioned above, the commercially available fenofibrate product is TRICOR® in the form of tablets or, alternatively, TRICOR® in the form of capsules.

A convenient method for determining whether a suitable amount of fenofibrate has been absorbed may be to determine the content of unchanged fibrate excreted via the feces. Thus, in one embodiment the invention relates to a solid pharmaceutical composition or solid dosage form, wherein at most about 25% w/w such as, e.g., at the most about 20% w/w, at the most about 15% w/w, at the most about 10% w/w, at the most about 5% w/w of the fenofibrate contained in the composition is excreted in the feces after oral administration.

The Vehicle

Vehicles useful in the present context are vehicles, which are water-miscible, hydrophilic or hydrophobic. Useful vehicles are non-aqueous substances which may be hydrophilic, lipophilic, hydrophobic and/or amphiphilic materials. The hydrophobic or hydrophilic or water-miscible vehicles will normally be liquid at ambient or elevated temperature. In the present context the term “a hydrophobic or a hydrophilic or water-miscible vehicle” is used in a very broad sense including oils, waxes, semi-solid materials and materials that normally are used as solvents (such as organic solvents) or cosolvents within the pharmaceutical industry, and the term also includes therapeutically and/or prophylactically active substances that are in liquid form at ambient temperature; furthermore the term includes emulsions like e.g., microemulsions and nanoemulsions and suspensions.

The hydrophobic or hydrophilic or water-miscible vehicles that are suitable for use in the present context are substances or materials, which have a melting point of at least about 0° C. and at the most about 250° C.

Interesting hydrophobic or hydrophilic or water-miscible vehicles are generally substances, which are used in the manufacture of pharmaceuticals as so-called melt binders or solid solvents (in the form of solid dosage form), or as co-solvents or ingredients in pharmaceuticals for topical use.

It may be hydrophilic, hydrophobic and/or have surface-active properties. In general hydrophilic and/or hydrophobic vehicles are suitable for use in the manufacture of a particulate material or a solid dosage form according to the invention. In a specific embodiment they may be used when the release of the active substance from the pharmaceutical composition is designed to be immediate or non-modified or modified. Hydrophobic vehicles are normally used in the manufacture of a modified release pharmaceutical composition. These considerations are simplified to illustrate general principles, but there are many cases where other combinations of vehicles and other purposes are relevant and, therefore, the examples above should not in any way limit the invention.

Examples of hydrophobic vehicles useful in the present invention are straight chain saturated hydrocarbons, paraffins; fats and oils such as cacao butter, beef tallow, lard; higher fatty acid such as stearic acid, myristic acid, palmitic acid; hydrogenated tallow, substituted and/or unsubstituted triglycerides, yellow beeswax, white beeswax, carnauba wax, castor wax, japan wax, and mixtures thereof.

Examples of water-miscible vehicles useful in the present invention are water-miscible polar lipids such as sorbitan esters, polyether glycol esters; higher alcohols such as cetanol, stearyl alcohol; glyceryl monooleate, substituted and/or unsubstituted monoglycerides, substituted and/or unsubstituted diglycerides, and mixtures thereof. In a more preferred embodiment, the vehicle is hydrophilic or water-miscible. Preferably, the vehicle is selected from the group consisting of polyethylene glycols, polyoxyethylene oxides, poloxamers, polyoxyethylene stearates, poly-epsilon caprolactone and mixtures thereof. However, the vehicle may advantageously also be a polyglycolized glyceride such as one of the numerous products sold under the registered trade mark GELUCIRE®, for example Gelucire 44/14.

Examples of useful hydrophilic or water-miscible vehicles are polyvinylpyrrolidones, polyvinyl-polyvinylacetate copolymers (PVP-PVA), polyvinyl alcohol (PVA), PVP polymers, acrylic polymers, polymethacrylic polymers (Eudragit RS; Eudragit RL, Eudragit NE, Eudragit E), myristyl alcohol, cellulose derivatives including hydroxypropyl methylcellulose (HPMC), hydroxypropyl cellulose (HPC), methylcellulose, sodium carboxymethylcellulose, hydroxyethyl cellulose, pectins, cyclodextrins, galactomannans, alginates, carragenates, xanthan gums and mixtures thereof.

The vehicle is preferably a mixture of two or more substances.

The vehicle may also be an oily material as defined and described below. Preferably, the melting point of the vehicle is preferably in the range of 10° C. to 250° C., preferably in the range of 30° C. to 100° C., more preferably in the range of 40° C. to 75° C., especially in the range of 40° C. to 70° C. In specific embodiments of the invention, the hydrophobic or hydrophilic or water-miscible vehicles have a melting point of about 5° C. or more such as, e.g., about 10° C. or more, about 15° C. or more, about 20° C. or more or about 25° C. or more. Normally, vehicles having such a low melting point require addition of an oil-sorption material. However, a person skilled in the art will know when it is necessary to add such an oil-sorption material.

In the present context, melting points are determined by DSC (Differential Scanning Calorimetry). The melting point is determined as the temperature at which the linear increase of the DSC curve intersects the temperature axis.

In an interesting embodiment, the vehicle is a polyethylene glycol having an average molecular weight in a range of from about 400 to about 35,000 such as, e.g., from about 800 to about 35,000, from about 1,000 to about 35,000 such as, e.g., polyethylene glycol 1,000, polyethylene glycol 2,000, polyethylene glycol 3,000, polyethylene glycol 4,000, polyethylene glycol 5,000, polyethylene glycol 6000, polyethylene glycol 7,000, polyethylene glycol 8,000, polyethylene glycol 9,000 polyethylene glycol 10,000, polyethylene glycol 15,000, polyethylene glycol 20,000, or polyethylene glycol 35,000. In certain situations polyethylene glycol may be employed with a molecular weight from about 35,000 to about 100,000.

In another interesting embodiment, the vehicle is polyethylene oxide having a molecular weight of from about 2,000 to about 7,000,000 such as, e.g. from about 2,000 to about 100,000, from about 5,000 to about 75,000, from about 10,000 to about 60,000, from about 15,000 to about 50,000, from about 20,000 to about 40,000, from about 100,000 to about 7,000,000 such as, e.g., from about 100,000 to about 1,000,000, from about 100,000 to about 600,000, from about 100,000 to about 400,000 or from about 100,000 to about 300,000.

In another embodiment, the vehicle is a poloxamer such as, e.g., Poloxamer 188, Poloxamer 237, Poloxamer 338 or Poloxamer 407 or other block copolymers of ethylene oxide and propylene oxide such as the PLURONIC® and/or Tetronic® series. Suitable block copolymers of the PLURONIC® series include polymers having a molecular weight of about 3,000 or more such as, e.g., from about 4,000 to about 20,000 and/or a viscosity (Brookfield) from about 200 to about 4,000 cps such as, e.g., from about 250 to about 3,000 cps. Suitable examples include PLURONIC® F38, P65, P68LF, P75, F77, P84, P85, F87, F88, F98, P103, P104, P105, F108, P123, F123, F127, 10R8, 17R8, 25R5, 25F8 etc. Suitable block copolymers of the TETRONIC® series include polymers having a molecular weight of about 8,000 or more such as, e.g., from about 9,000 to about 35,000 and/or a viscosity (Brookfield) of from about 500 to about 45,000 cps such as, e.g., from about 600 to about 40,000. The viscosities given above are determined at 60° C. for substances that are pastes at room temperature and at 77° C. for substances that are solids at room temperature.

In a specific embodiment a particulate material according to the invention comprises as vehicle a mixture of a polyethylene glycol and a poloxamer in a proportion (weight) of between about 1:3 and about 10:1, preferably between about 1:1 and about 5:1, more preferably between about 3:2 and about 4:1, especially between about 2:1 and about 3:1, in particular about 7:3.

In particular the poloxamer is poloxamer 188.

In another embodiment, polyethylene glycol is employed as a vehicle and it has an average molecular weight of about 6000 (PEG6000).

The vehicle may also be a sorbitan ester such as, e.g., sorbitan di-isostearate, sorbitan dioleate, sorbitan monolaurate, sorbitan monoisostearate, sorbitan monooleate, sorbitan monopalmitate, sorbitan monostearate, sorbitan sesqui-isostearate, sorbitan sesquioleate, sorbitan sesquistearate, sorbitan tri-isostearate, sorbitan trioleate, sorbitan tristearate or mixtures thereof.

The vehicle may also comprise a mixture of different vehicles such as, e.g., a mixture of hydrophilic and/or hydrophobic materials.

Other suitable vehicles may be solvents or semi-solid excipients like, e.g. propylene glycol, polyglycolised glycerides including Gelucire 44/14, complex fatty materials of plant origin including theobroma oil, carnauba wax, vegetable oils like e.g. almond oil, coconut oil, corn oil, cottonseed oil, sesame oil, soya oil, olive oil, castor oil, palm kernels oil, peanut oil, rape oil, grape seed oil etc., hydrogenated vegetable oils such as, e.g. hydrogenated peanut oil, hydrogenated palm kernels oil, hydrogenated cottonseed oil, hydrogenated soya oil, hydrogenated castor oil, hydrogenated coconut oil; natural fatty materials of animal origin including beeswax, lanolin, fatty alcohols including cetyl, stearyl, lauric, myristic, palmitic, stearic fatty alcohols; esters including glycerol stearate, glycol stearate, ethyl oleate, isopropyl myristate; liquid interesterified semi-synthetic glycerides including Miglycol 810/812; amide or fatty acid alcolamides including stearamide ethanol, diethanolamide of fatty coconut acids, acetic acid esters of mono and di-glycerides, citric acid esters of mono and di-glycerides, lactic acid esters of mono and diglycerides, mono and di-glycerides, poly-glycerol esters of fatty acids, poly-glycerol poly-ricinoleate, propylene glycol esters of fatty acids, sorbitan monostearates, sorbitan tristearates, sodium stearoyl lactylates, calcium stearoyl lactylates, diacetyl tartaric acid esters of mono and di-glycerides etc.

One of the advantages is that is it possible to incorporate a relatively large amount of vehicle and still have a material that is solid. Thus, it is possible to prepare solid compositions with a relatively high load of vehicle by use of an oil sorption material as mentioned above. Within the pharmaceutical field it is an advantage to be able to incorporate a relatively large amount of a vehicle (e.g., with oil or oily-like characteristics) in a solid composition especially in those situation where the active substance does not have suitable properties with respect to water solubility (e.g., poor water solubility), stability in aqueous medium (i.e. degradation occurs in aqueous medium), oral bioavailability (e.g. low bioavailability) etc., or in those situations where it is desired to modify the release of an active substance from a composition in order to obtain a controlled, modified, delayed, sustained and/or pulsed delivery of the active substance.

It is within the skills of the average practioner to select a suitable vehicle being pharmaceutical acceptable, capable of dispersing, dissolving or at least partly dissolving the active substances and having a melting point in the desired range using general knowledge and routine experimentation. Suitable candidate for vehicles are described in WO 03/004001, which is herein incorporated by reference.

In the present context, suitable vehicle are e.g. those mentioned above as well as those disclosed in WO 03/004001.

Particulate Material

The particulate material according to the invention has a suitable flowability as determined according to the method described in Ph.Eur. measuring the flow rate of the composition out of a funnel with a nozzle diameter of 10.0 mm. In order to avoid any adherence to the manufacturing and/or filling equipment it is important that the particulate material is free-flowing. This characteristic is also important in those cases where it is desired to process the particulate material further into other kinds of formulations such as, e.g., solid dosage forms.

When the particulate material is a free-flowing powder it can be immediately processed into e.g. solid dosage forms such as tablets, capsules or sachets. Normally, the particulate material has properties that are suitable in order to manufacture tablets by direct compression without addition of large amounts of further additives.

As mentioned above, the particulate material according to the invention contains a vehicle. In some embodiments this vehicle is an oily material which may be present in a relatively high amount. In such cases it may be necessary to include in the material a substance that has adsorbing or absorbing properties so that the final particulate material appears as a non-oily powder and not during storage release some of the vehicle that could result in a oily surface. Accordingly, the particulate material may contain one or more oil-sorption materials, which—when tested as described herein
i) has an oil threshold value of 10% or more, when tested according to the Threshold Test herein, and at least one of
ii) releases at least 30% of an oil, when tested according to the Release Test herein, and
iii) in the form of a tablet has a disintegration time of at the most 1 hour, when tested according to Ph. Eur. Disintegration test, the tablet containing about 90% w/w or more of the oil-sorption material. In certain situations, it has been found that it is an advantage to incorporate a sorption material in the composition in order e.g., to enable a high concentration of a vehicle has oil or oily-like character. In those cases where the vehicle has a melting point of at the most about 25° C., it may be especially suitable to incorporate a sorption material. Suitable examples of materials suitable as vehicles as well as sorption materials are given herein.
   Pharmaceutically Acceptable Excipients and Additives

In the present context the terms “pharmaceutically acceptable excipient” are intended to denote any material, which is inert in the sense that it substantially does not have any therapeutic and/or prophylactic effect per se. Such an excipient may be added with the purpose of making it possible to obtain a pharmaceutical, cosmetic and/or foodstuff composition, which have acceptable technical properties. A particulate material or a solid dosage form according to the invention may contain one or more pharmaceutically acceptable excipients.

Examples of suitable excipients for use in a composition or solid dosage form according to the invention include fillers, diluents, disintegrants, binders, lubricants etc. or mixture thereof. As the composition or solid dosage form according to the invention may be used for different purposes, the choice of excipients is normally made taken such different uses into considerations. Other pharmaceutically acceptable excipients for suitable use are e.g. acidifying agents, alkalizing agents, preservatives, antioxidants, buffering agents, chelating agents, coloring agents, complexing agents, emulsifying and/or solubilizing agents, flavors and perfumes, humectants, sweetening agents, wetting agents etc.

Examples of suitable fillers, diluents and/or binders include lactose (e.g. spray-dried lactose, α-lactose, β-lactose, TABLETOSE®, various grades of PHARMATOSE®, MICROTOSE® or FAST-FLOC®), microcrystalline cellulose (various grades of AVICEL®, ELCEMA®, VIVACEL®, MING TAI® or SOLKA-FLOC®), hydroxypropylcellulose, L-hydroxypropylcellulose (low substituted), hydroxypropyl methylcellulose (HPMC) (e.g., Methocel E, F and K, Metolose SH of Shin-Etsu, Ltd, such as, e.g. the 4,000 cps grades of Methocel E and Metolose 60 SH, the 4,000 cps grades of Methocel F and Metolose 65 SH, the 4,000, 15,000 and 100,000 cps grades of Methocel K; and the 4,000, 15,000, 39,000 and 100,000 grades of Metolose 90 SH), methylcellulose polymers (such as, e.g., Methocel A, Methocel A4C, Methocel A15C, Methocel A4M), hydroxyethylcellulose, sodium carboxymethylcellulose, carboxymethylene, carboxymethylhydroxyethylcellulose and other cellulose derivatives, sucrose, agarose, sorbitol, mannitol, dextrins, maltodextrins, starches or modified starches (including potato starch, maize starch and rice starch), calcium phosphate (e.g., basic calcium phosphate, calcium hydrogen phosphate, dicalcium phosphate hydrate), calcium sulfate, calcium carbonate, sodium alginate, collagen etc.

Specific examples of diluents are e.g., calcium carbonate, dibasic calcium phosphate, tribasic calcium phosphate, calcium sulfate, microcrystalline cellulose, powdered cellulose, dextrans, dextrin, dextrose, fructose, kaolin, lactose, mannitol, sorbitol, starch, pregelatinized starch, sucrose, sugar etc.

Specific examples of disintegrants are e.g. alginic acid or alginates, microcrystalline cellulose, hydroxypropyl cellulose and other cellulose derivatives, croscarmellose sodium, crospovidone, polacrillin potassium, sodium starch glycolate, starch, pregelatinized starch, carboxymethyl starch (e.g. PRIMOGEL® and EXPLOTAB®) etc.

Specific examples of binders are e.g., acacia, alginic acid, agar, calcium carrageenan, sodium carboxymethylcellulose, microcrystalline cellulose, dextrin, ethylcellulose, gelatin, liquid glucose, guar gum, hydroxypropyl methylcellulose, methylcellulose, pectin, PEG, povidone, pregelatinized starch etc.

Glidants and lubricants may also be included in the second composition. Examples include stearic acid, magnesium stearate, calcium stearate or other metallic stearate, talc, waxes and glycerides, light mineral oil, PEG, glyceryl behenate, colloidal silica, hydrogenated vegetable oils, corn starch, sodium stearyl fumarate, polyethylene glycols, alkyl sulfates, sodium benzoate, sodium acetate etc.

Other excipients which may be included in a composition or solid dosage form of the invention are e.g., flavoring agents, coloring agents, taste-masking agents, pH-adjusting agents, buffering agents, preservatives, stabilizing agents, anti-oxidants, wetting agents, humidity-adjusting agents, surface-active agents, suspending agents, absorption enhancing agents, agents for modified release etc.

Other additives in a composition or a solid dosage form according to the invention may be antioxidants like e.g. ascorbic acid, ascorbyl palmitate, butylated hydroxyanisole, butylated hydroxytoluene, hypophosphorous acid, monothioglycerol, potassium metabisulfite, propyl gallate, sodium formaldehylde sulfoxylate, sodium metabisulfite, sodium thiosulfate, sulfur dioxide, tocopherol, tocopherol acetate, tocopherol hemisuccinate, TPGS or other tocopherol derivatives, etc. The carrier composition may also contain e.g., stabilising agents. The concentration of an antioxidant and/or a stabilizing agent in the carrier composition is normally from about 0.1% w/w to about 5% w/w.

A composition or solid dosage form according to the invention may also include one or more surfactants or substances having surface-active properties. It is contemplated that such substances are involved in the wetting of the slightly soluble active substance and thus, contributes to improved solubility characteristics of the active substance. Suitable surfactants for use in a composition or a solid dosage form according to the invention are surfactants such as, e.g., hydrophobic and/or hydrophilic surfactants as those disclosed in WO 00/50007 in the name of Lipocine, Inc.

Specific examples of suitable surfactants are polyethoxylated fatty acids such as, e.g., fatty acid mono- or diesters of polyethylene glycol or mixtures thereof such as, e.g., mono- or diesters of polyethylene glycol with lauric acid, oleic acid, stearic acid, myristic acid, ricinoleic acid, and the polyethylene glycol may be selected from PEG 4, PEG 5, PEG 6, PEG 7, PEG 8, PEG 9, PEG 10, PEG 12, PEG 15, PEG 20, PEG 25, PEG 30, PEG 32, PEG 40, PEG 45, PEG 50, PEG 55, PEG 100, PEG 200, PEG 400, PEG 600, PEG 800, PEG 1000, PEG 2000, PEG 3000, PEG 4000, PEG 5000, PEG 6000, PEG 7000, PEG 8000, PEG 9000, PEG 1000, PEG 10,000, PEG 15,000, PEG 20,000, PEG 35,000, polyethylene glycol glycerol fatty acid esters, i.e. esters like the above-mentioned but in the form of glyceryl esters of the individual fatty acids; glycerol, propylene glycol, ethylene glycol, PEG or sorbitol esters with e.g., vegetable oils like e.g., hydrogenated castor oil, almond oil, palm kernel oil, castor oil, apricot kernel oil, olive oil, peanut oil, hydrogenated palm kernel oil and the like, polyglycerized fatty acids like e.g., polyglycerol stearate, polyglycerol oleate, polyglycerol ricinoleate, polyglycerol linoleate, propylene glycol fatty acid esters such as, e.g., propylene glycol monolaurate, propylene glycol ricinoleate and the like, mono- and diglycerides like e.g. glyceryl monooleate, glyceryl dioleae, glyceryl mono- and/or dioleate, glyceryl caprylate, glyceryl caprate etc.; sterol and sterol derivatives; polyethylene glycol sorbitan fatty acid esters (PEG-sorbitan fatty acid esters) such as esters of PEG with the various molecular weights indicated above, and the various TWEEN® series (from ICI America, Inc.); polyethylene glycol alkyl ethers such as, e.g., PEG oleyl ether and PEG lauryl ether; sugar esters like e.g. sucrose monopalmitate and sucrose monolaurate; polyethylene glycol alkyl phenols like e.g. the TRITON® X or N series (Union Carbide Chemicals & Plastics Technology Corporation); polyoxyethylene-polyoxypropylene block copolymers such as, e.g., the PLURONIC® series from BASF Aktiengesellschaft, the SYNPERONIC® series from ICI America, Inc., Emkalyx, LUTROL® from BASF Aktiengesellschaft, Supronic etc. The generic term for these polymers is “poloxamers” and relevant examples in the present context are Poloxamer 105, 108, 122, 123, 124, 181, 182, 183, 184, 185, 188, 212, 215, 217, 231, 234, 235, 237, 238, 282, 284, 288, 331, 333, 334, 335, 338, 401, 402, 403 and 407; sorbitan fatty acid esters like the SPAN® series (from ICI) or ARLACEL® series (from ICI) such as, e.g., sorbitan monolaurate, sorbitan monopalmitate, sorbitan monooleate, sorbitan monostearate etc.; lower alcohol fatty acid esters like e.g., oleate, isopropyl myristate, isopropyl palmitate etc.; ionic surfactants including cationic, anionic and zwitterionic surfactants such as, e.g., fatty acid salts, bile salts, phospholipids, phosphoric acid esters, carboxylates, sulfates and sulfonates etc.

When a surfactant or a mixture of surfactants is present in a composition or a solid dosage form of the invention, the concentration of the surfactant(s) is normally in a range of from about 0.1-80% w/w such as, e.g., from about 0.1 to about 20% w/w, from about 0.1 to about 15% w/w, from about 0.5 to about 10% w/w, or alternatively, from about 0.10 to about 80% w/w such as, e.g. from about 10 to about 70% w/w, from about 20 to about 60% w/w or from about 30 to about 50% w/w.

In a specific aspect of the invention, the at least one of the one or more pharmaceutically acceptable excipient is selected from the group consisting of silica acid or a derivative or salt thereof including silicates, silicon dioxide and polymers thereof; magnesium aluminosilicate and/or magnesium aluminometasilicate, bentonite, kaolin, magnesium trisilicate, montmorillonite and/or saponite.

Sorption Materials

Materials such as those mentioned immediately above are especially useful as a sorption material for oily materials in pharmaceuticals, cosmetics and/or foodstuff. In a specific embodiment, the material is used as a sorption material for oily materials in pharmaceuticals. The material that has the ability to function as a sorption material for oily materials is also denoted “oil sorption material”.

Furthermore, in the present context the term “sorption” is used to denote “absorption” as well as “adsorption”. It should be understood that whenever one of the terms is used it is intended to cover the phenomenon absorption as well as adsorption. The terms “sorption material” and “oil sorption material” is intended to have the same meaning.

A sorption material suitable for use according to the present invention is a solid pharmaceutically acceptable material, which—when tested as described herein
i) has an oil threshold value of 10% or more, when tested according to the Threshold Test disclosed herein, and which material is used in a composition of the invention further fulfilling one or both of i) and ii):
i) the compositon releases at least 30% of the hydrophobic or a hydrophilic or water-miscible vehicle, when tested according to the Release Test;
ii) the composition, in the form of a tablet, contains at least about 90% w/w of the oil-sorption material, and exhibits a disintegration time of at the most 60 minutes when tested according to the Ph. Eur. Disintegration Test.

The material is especially useful as a sorption material for oily materials in pharmaceuticals, cosmetics and/or foodstuff, especially in pharmaceuticals.

It is important that the oil sorption material fulfills at least two tests. One of the tests is mandatory, i.e. the Threshold Test must be met. This test gives a measure for how much oily material the oil sorption material is able to absorb while retaining suitable flowability properties. It is important that an oil sorption material for use according to the invention (with or without oil absorbed) has a suitable flowability so that it easily can be admixed with other excipients and/or further processed into compositions without significant problems relating to e.g. adherence to the apparatus involved. The test is described below in Materials and Methods and guidance is given for how the test is carried out. The Threshold Test involves the determination of the flowability of the solid material loaded with different amounts of oil.

From above it is seen that the oil threshold value normally must exceed 10% and often the oil sorption material has an oil threshold value of at least about 15%, such as, e.g., at least about 20%, at least about 25%, at least about 30%, at least about 35%, at least about 40%, or at least about 45%.

An especially suitable material for use according to the invention, Aeropearl 300, has a very high oil threshold value of about 60%. Accordingly, materials that have an oil threshold value of at least about 50%, such as, e.g., at least about 55% or at least about 60% are used in specific embodiments of the present invention.

Furthermore, an oil sorption material for use according to the invention must fulfill at least one further test, namely a release test and/or a disintegration test.

The release test gives a measure of the ability of an oil sorption material to release the oil that is absorbed to the material when contacted with water. This ability is very important especially in those situations where an active substance is contained in the oily material. If the oil sorption material is not capable of releasing the oil from the material then there is a major risk that the active substance will only to a minor degree be released from the material. Accordingly, it is envisaged that bioavailability problems relating to e.g., poor absorption etc. will occur in such situations.

The requirements for the release test are that the solid pharmaceutical acceptable material, when tested as described herein, releases at least about 30% such as, e.g., at least about 35%, at least about 40%, at least about 45%, at least about 50%, at least about 55% or at least about 60% of an oil. As it appears from the examples herein a suitable oil sorption material like Aeroperl 300 has a much higher release. Therefore, in a specific embodiment of the invention, the solid pharmaceutical acceptable material, when tested as described herein, releases at least about 65% such as, e.g., at least about 70%, at least about 75% or at least about 80% of an oil.

The disintegration test is not performed on the solid material in particulate form but on a tablet made of the solid material. A requirement with respect to disintegration is important in order to ensure that the solid material, when included in solid dosage forms, does not impart unwanted properties to the dosage form e.g., leading to unwanted properties with respect to dissolution and bioavailability of the active substance contained in the dosage form. For some of the materials suitable for use according to the invention it is possible to press tablets containing 100% w/w of the solid material itself. If this is the case, the test is carried out on such tablets. However, it is envisaged that there may be situations where it is rather difficult to prepare tablets from the solid material alone. In such cases it is possible to add pharmaceutically acceptable excipients normally used in the preparation of compressed tablets up to a concentration of 10% w/w or less. Examples of suitable pharmaceutically acceptable excipients include fillers, diluents, binders and lubricants. However, excipients, normally classified as disintegrants, should be avoided.

Accordingly, the solid pharmaceutical acceptable material for use according to invention, when tested as described herein, in the form of a tablet should have a disintegration time of at the most 1 hour, when tested according to Ph. Eur. Disintegration test, the tablet containing about 90% w/w or more, such as, e.g., about 92.5% w/w or more, about 95% w/w or more, about 97.5% w/w or more or about 100% of the pharmaceutically acceptable material.

In a further embodiment, the solid pharmaceutical acceptable material, when tested as described herein, in the form of a tablet has a disintegration time of at the most about 50 min, such as, e.g., at the most about 40 min, at the most about 30 min, at the most about 20 min, at the most about 10 min or at the most about 5 min, when tested according to Ph. Eur. Disintegration test, the tablet containing about 90% w/w or more, such as, e.g., about 92.5% w/w or more, about 95% w/w or more, about 97.5% w/w or more or about 100% of the pharmaceutically acceptable material.

In a specific embodiment, the solid material used as a sorption material fulfils all three tests. Thus, the solid pharmaceutical acceptable material, when tested as described herein,
i) has an oil threshold value of at least about 10%, such as, e.g., at least about 15%, at least about 20%, at least about 25%, at least about 30%, at least about 35%, at least about 40%, at least about 45%, at least about 50%, at least about 55% or at least about 60%,
ii) releases at least about 30% such as, e.g., at least about 35%, at least about 40%, at least about 45%, at least about 50%, at least about 55%, at least about 60%, at least about 65%, at least about 70%, at least about 75% or at least about 80% of an oil, and
iii) in the form of a tablet has a disintegration time of at the most 1 hour such as at the most about 50 min, at the most about 40 min, at the most about 30 min, at the most about 20 min, at the most about 10 min or at the most about 5 min, when tested according to Ph. Eur. Disintegration test, the tablet containing about 90% w/w or more, such as, e.g., about 92.5% w/w or more, about 95% w/w or more, about 97.5% w/w or more or about 100% of the pharmaceutically acceptable material.

Other specific embodiments of the invention are those, wherein the solid pharmaceutical material used as a sorption material in a composition according ot the invention, when tested as described herein,
i) has an oil threshold value of at least about 55%; the solid pharmaceutical material, when tested as described herein,
ii) releases at least about 75% of an oil; and/or the solid pharmaceutical material, when tested as described herein,
iii) in the form of a tablet has disintegration time of at the most about 10 min, when tested according to Ph. Eur. Disintegration test, the tablet containing about 97.5% w/w of the pharmaceutically acceptable material.

The solid pharmaceutically acceptable material used as a sorption material in a composition according to the invention is normally a particulate material in the form of e.g. powders, particles, granules, granulates etc.

Such particulate material suitable for use as an oil sorption material has normally a bulk density of about 0.15 g/cm³ or more such as, e.g., at least about 0.20 g/cm³ or at least about 0.25 g/cm³.

Furthermore, the oil sorption material normally has an oil absorption value of at least about 100 g oil/100 g such as, e.g., at least about 150 g oil/100 g, at least about 200 g oil/100 g, at least about 250 g oil/100 g, at least about 300 g oil/100 g or at least about 400 g oil/100 g pharmaceutically acceptable material. The oil absorption value is determined as described in the experimental section herein.

The present inventors have found that a common feature of some of the materials suitable for use as oil sorption material is that they have a relatively large surface area. Accordingly, pharmaceutically acceptable material for use as an oil sorption material according to the invention may have a BET surface area of at least 5 m²/g such as, e.g., at least about 25 m²/g, at least about 50 m²/g, at least about 100 m²/g, at least about 150 m²/g, at least about 200 m²/g, at least about 250 m²/g or at least about 275 m²/g.

As mentioned above one of the characteristic features of a pharmaceutically acceptable material for use as an oil sorption material according to the invention is that it retains a good flowability even if it has been loaded with oily material. Thus, the flowability of the pharmaceutically acceptable material loaded with about 25% w/w or more such as, e.g. about 30% w/w or more, about 40% w/w or more, about 45% w/w or more, about 50% w/w or more, about 55% w/w or more, about 60% w/w or more, about 65% w/w or more or about about 70% w/w viscoleo will normally meet the Ph. Eur. requirements.

Notably, the oil sorption material may comprise a silica acid or a derivative or salt thereof such as, e.g., silicon dioxide or a polymer thereof as a pharmaceutically acceptable excipient. However, dependent on the quality employed a silicon dioxide may be a lubricant or it may be an oil sorption material. Qualities fulfilling the latter function seem to be most important.

In a specific embodiment, a composition or solid dosage form according to invention comprises a pharmaceutically acceptable excipient that is a silicon dioxide product that has properties corresponding to AEROPERL® 300

Use of an oil sorption material in compositions or dosage forms according to the invention is very advantageous for the preparation of pharmaceutical, cosmetic, nutritional and/or food compositions, wherein the composition comprises oily material. One of the advantages is that is it possible to incorporate a relatively large amount of and oily material and still have a material that is solid. Thus, it is possible to prepare solid compositions with a relatively high load of oily materials by use of an oil sorption material according to the invention. Within the pharmaceutical field it is an advantage to be able to incorporate a relatively large amount of an oily material in a solid composition especially in those situation where the active substance does not have suitable properties with respect to water solubility (e.g. poor water solubility), stability in aqueous medium (i.e. degradation occurs in aqueous medium), oral bioavailability (e.g. low bioavailability) etc., or in those situations where it is desired to modify the release of an active substance from a composition in order to obtain a controlled, delayed, sustained and/or pulsed delivery of the active substance. Thus, in a specific embodiment it is used in the preparation of pharmaceutical compositions.

The oil sorption material for use in the processing into solid compositions normally absorbs about 5% w/w or more, such as, e.g., about 10% w/w or more, about 15% w/w or more, about 20% w/w or more, about 25% w/w or more, about 30% w/w or more, about 35% w/w or more, about 40% w/w or more, about 45% w/w or more, about 50 w/w or more, about 55% w/w or more, about 60% w/w or more, about 65% w/w or more, about 70% w/w or more, about 75% w/w or more, about 80% w/w or more, about 85% w/w or more, about 90% w/w or more or about 95% w/w or more of an oil or an oily material and is still a solid material.

Oily Materials

An important aspect of the invention is compositions or solid dosage forms comprising an oily material.

In the present context the term “oily materials” is used in a very broad sense including oils, waxes, semi-solid materials and materials that normally are used as solvents (such as organic solvents) or cosolvents within the pharmaceutical industry, and the term also includes therapeutically and/or prophylactically active substances that are in liquid form at ambient temperature; furthermore the term includes emulsions like e.g. microemulsions and nanoemulsions and suspensions. The oils and oily-like materials that can be absorbed are normally liquid at ambient or elevated temperature (for practical reasons the max. temperature is about 250° C.). They may be hydrophilic, lipophilic, hydrophobic and/or amphiphilic materials.

The oily materials that are suitable for use in the present context are substances or materials, which have a melting point of at least about 10° C. and at the most about 250° C.

In specific embodiments of the invention, the oily material has a melting point of about 5° C. or more such as, e.g., about 10° C. or more, about 15° C. or more, about 20° C. or more or about 25° C. or more.

In further embodiments of the invention, the oily material has a melting point of at least about 25° C. such as, e.g., at least about 30° C. at least about 35° C. or at least about 40° C. For practical reasons, the melting point may normally not be too high, thus, the oily material normally has a melting point of at the most about 250° C., at the most about 200° C., at the most about 150° C. or at the most about 100° C. If the melting point is higher a relatively high temperature may promote e.g. oxidation or other kind of degradation of an active substance in those cases where e.g. a therapeutically and/or prophylactically active substance is included.

Interesting oily materials are in general substances, which are used in the manufacture of pharmaceuticals as so-called melt binders or solid solvents (in the form of solid dosage form), or as co-solvents or ingredients in pharmaceuticals for topical use. It may be hydrophilic, hydrophobic and/or have surface-active properties. In general hydrophilic and/or hydrophobic oily materials are suitable for use in the manufacture of a pharmaceutical composition comprising a therapeutically and/or prophylactically active substance that has a relatively low aqueous solubility and/or when the release of the active substance from the pharmaceutical composition is designed to be immediate or non-modified. Hydrophobic oily materials, on the other hand, are normally used in the manufacture of a modified release pharmaceutical composition. The above-given considerations are simplified to illustrate general principles, but there are many cases where other combinations of oily materials and other purposes are relevant and, therefore, the examples above should not in any way limit the invention.

Typically, a suitable hydrophilic oily material is selected from the group consisting of: polyether glycols such as, e.g., polyethylene glycols, polypropylene glycols; polyoxyethylenes; polyoxypropylenes; poloxamers and mixtures thereof, or it may be selected from the group consisting of: xylitol, sorbitol, potassium sodium tartrate, sucrose tribehenate, glucose, rhamnose, lactitol, behenic acid, hydroquinon monomethyl ether, sodium acetate, ethyl fumarate, myristic acid, citric acid, Gelucire 50/13, other Gelucire types such as, e.g., Gelucire 44/14 etc., Gelucire 50/10, Gelucire 62/05, Sucro-ester 7, Sucro-ester 11, Sucro-ester 15, maltose, mannitol and mixtures thereof.

A suitable hydrophobic oily material may be selected from the group consisting of: straight chain saturated hydrocarbons, sorbitan esters, paraffins; fats and oils such as e.g., cacao butter, beef tallow, lard, polyether glycol esters; higher fatty acid such as, e.g. stearic acid, myristic acid, palmitic acid, higher alcohols such as, e.g., cetanol, stearyl alcohol, low melting point waxes such as, e.g., glyceryl monostearate, glyceryl monooleate, hydrogenated tallow, myristyl alcohol, stearyl alcohol, substituted and/or unsubstituted monoglycerides, substituted and/or unsubstituted diglycerides, substituted and/or unsubstituted triglycerides, yellow beeswax, white beeswax, carnauba wax, castor wax, japan wax, acetylate monoglycerides; NVP polymers, PVP polymers, acrylic polymers, or a mixture thereof.

In an interesting embodiment, the oily material is a polyethylene glycol having an average molecular weight in a range of from about 400 to about 35,000 such as, e.g., from about 800 to about 35,000, from about 1,000 to about 35,000 such as, e.g., polyethylene glycol 1,000, polyethylene glycol 2,000, polyethylene glycol 3,000, polyethylene glycol 4,000, polyethylene glycol 5,000, polyethylene glycol 6000, polyethylene glycol 7,000, polyethylene glycol 8,000, polyethylene glycol 9,000 polyethylene glycol 10,000, polyethylene glycol 15,000, polyethylene glycol 20,000, or polyethylene glycol 35,000. In certain situations polyethylene glycol may be employed with a molecular weight from about 35,000 to about 100,000.

In another interesting embodiment, the oily material is polyethylene oxide having a molecular weight of from about 2,000 to about 7,000,000 such as, e.g. from about 2,000 to about 100,000, from about 5,000 to about 75,000, from about 10,000 to about 60,000, from about 15,000 to about 50,000, from about 20,000 to about 40,000, from about 100,000 to about 7,000,000 such as, e.g., from about 100,000 to about 1,000,000, from about 100,000 to about 600,000, from about 100,000 to about 400,000 or from about 100,000 to about 300,000.

In another embodiment, the oily material is a poloxamer such as, e.g. Poloxamer 188, Poloxamer 237, Poloxamer 338 or Poloxamer 407 or other block copolymers of ethylene oxide and propylene oxide such as the PLURONIC® and/or TETRONIC® series. Suitable block copolymers of the PLURONIC® series include polymers having a molecular weight of about 3,000 or more such as, e.g. from about 4,000 to about 20,000 and/or a viscosity (Brookfield) from about 200 to about 4,000 cps such as, e.g., from about 250 to about 3,000 cps. Suitable examples include PLURONIC® (BASF) F38, P65, P68LF, P75, F77, P84, P85, F87, F88, F98, P103, P104, P105, F108, P123, F123, F127, 10R8, 17R8, 25R5, 25R8 etc. Suitable block copolymers of the TETRONIC® series (BASF) include polymers having a molecular weight of about 8,000 or more such as, e.g., from about 9,000 to about 35,000 and/or a viscosity (Brookfield) of from about 500 to about 45,000 cps such as, e.g., from about 600 to about 40,000. The viscosities given above are determined at 60° C. for substances that are pastes at room temperature and at 77° C. for substances that are solids at room temperature.

The oily material may also be a sorbitan ester such as, e.g., sorbitan di-isostearate, sorbitan dioleate, sorbitan monolaurate, sorbitan monoisostearate, sorbitan monooleate, sorbitan monopalmitate, sorbitan monostearate, sorbitan sesqui-isostearate, sorbitan sesquioleate, sorbitan sesquistearate, sorbitan tri-isostearate, sorbitan trioleate, sorbitan tristearate or mixtures thereof.

The oily material may of course comprise a mixture of different oily materials such as, e.g., a mixture of hydrophilic and/or hydrophobic materials. Other suitable oily materials may be solvents or semi-solid excipients like, e.g., propylene glycol, polyglycolised glycerides including Gelucire 44/14, complex fatty materials of plant origin including theobroma oil, carnauba wax, vegetable oils like e.g., almond oil, coconut oil, corn oil, cottonseed oil, sesame oil, soya oil, olive oil, castor oil, palm kernels oil, peanut oil, rape oil, grape seed oil etc., hydrogenated vegetable oils such as, e.g., hydrogenated peanut oil, hydrogenated palm kernels oil, hydrogenated cottonseed oil, hydrogenated soya oil, hydrogenated castor oil, hydrogenated coconut oil; natural fatty materials of animal origin including beeswax, lanolin, fatty alcohols including cetyl, stearyl, lauric, myristic, palmitic, stearic fatty alcohols; esters including glycerol stearate, glycol stearate, ethyl oleate, isopropyl myristate; liquid interesterified semi-synthetic glycerides including Miglycol 810/812; amide or fatty acid alcolamides including stearamide ethanol, diethanolamide of fatty coconut acids, acetic acid esters of mono and di-glycerides, citric acid esters of mono and di-glycerides, lactic acid esters of mono and diglycerides, mono and di-glycerides, poly-glycerol esters of fatty acids, poly-glycerol poly-ricinoleate, propylene glycol esters of fatty acids, sorbitan monostearates, sorbitan tristearates, sodium stearoyl lactylates, calcium stearoyl lactylates, diacetyl tartaric acid esters of mono and di-glycerides etc.

The pharmaceutical composition or a solid dosage form according to the invention may have a concentration of the oily material in the composition or the dosage form of about 5% w/w or more such as, e.g., about 10% w/w or more, about 15% w/w or more, about 20% w/w or more, about 25% w/w or more, about 30% w/w or more, about 35% w/w or more, about 40% w/w or more, about 45% w/w or more, about 50 w/w or more, about 55% w/w or more, about 60% w/w or more, about 65% w/w or more, about 70% w/w or more, about 75% w/w or more, about 80% w/w or more, about 85% w/w or more, about 90% w/w or more or about 95% w/w or more.

In specific embodiments the concentration of the oily material in a composition or solid dosage form of the invention is in a range from about 20% to about 80% w/w such as, e.g., from about 25% to about 75% w/w.

One of the advantages is that is it possible to incorporate a relatively large amount of oily material and still have a solid material. Thus, it is possible to prepare solid compositions with a relatively high load of oily materials by use of an oil sorption material according to the invention. Within the pharmaceutical field it is an advantage to be able to incorporate a relatively large amount of an oily material in a solid composition especially in those situation where the active substance does not have suitable properties with respect to water solubility (e.g., poor water solubility), stability in aqueous medium (i.e. degradation occurs in aqueous medium), oral bioavailability (e.g., low bioavailability) etc., or in those situations where it is desired to modify the release of an active substance from a composition in order to obtain a controlled, delayed, sustained and/or pulsed delivery of the active substance.

Method of Manufacture

The particulate composition of the invention may be prepared by any method which is suitable for incorporation of poorly water-soluble active substances. The pharmaceutical compositions may be prepared by any convenient method such as, e.g. granulation, mixing, spray drying etc. A particularly useful method is the method disclosed in Applicants' co-pending international application published as WO 03/004001, which describes a process for preparation of particulate material by a controlled agglomeration method, i.e. a method, which enables a controlled growth in particle size. The method involves spraying a first composition comprising the active substance and a vehicle in liquid form onto a solid carrier. Normally, the vehicle has a melting point of at least 5° C., but the melting point must indeed be below the melting point of the active substance. In the present invention, the melting point of the vehicle and should not exceed 250° C.

It is within the skills of the average practitioner to select a suitable vehicle being pharmaceutical acceptable, capable of dispersing or fully or at least partly dissolving the active substance and having a melting point in the desired range using general knowledge and routine experimentation. Suitable candidate for carriers are described in WO 03/004001, which is herein incorporated by reference.

In the present context, suitable vehicles are e.g., those mentioned as vehicles or as oily materials as well as those disclosed in WO 03/004001. An advantage of using the controlled agglomeration method described in WO 03/004001 is that it is possible to apply a relatively large amount of a liquid system to a particulate material without having an undesirable growth in particle size. Accordingly, in one embodiment of the invention, the particulate material of a pharmaceutical composition has a geometric weight mean diameter d_{gw} of ≧10 μm such as, e.g. ≧20 [m, from about 20 to about 2000, from about 30 to about 2000, from about 50 to about 2000, from about 60 to about 2000, from about 75 to about 2000 such as, e.g. from about 100 to about 1500 μm, from about 100 to about 1000 μm or from about 100 to about 700 μm, or at the most about 400 μm or at the most 300 μm such as, e.g., from about 50 to about 400 μm such as, e.g., from about 50 to about 350 μm, from about 50 to about 300 μm, from about 50 to about 250 μm or from about 100 to about 300 μm.

The compositions and dosage forms of the invention are preferably formed by spray drying techniques, controlled agglomeration, freeze-drying or coating on carrier particles or any other solvent removal process. The dried product contains the active substance present preferably in dissolved form either fully dissolved as a solid solution or partly dissolved as a solid dispersion including a molecular dispersion and a solid solution.

However, the composition and dosage forms of the invention are preferably manufactured by a method comprising the steps of:
i) bringing the vehicle in liquid form, i.e. melting the vehicle if solid at room temperature,
ii) maintaining the liquid vehicle at a temperature below the melting point of the fibrate,
iii) dissolving the desired amount of fibrate in the vehicle,
iv) spraying the resulting solution onto a solid carrier having a temperature below the melting point of the vehicle,
v) mechanically working the resulting composition to obtain particles, i.e. a particulate material, and
vi) optionally subjecting the particulate material to conventional methods for preparing solid dosage forms.

Alternatively, the solid oral dosage form of the invention may be prepared by a method comprising the steps of
i) Bringing the vehicle in liquid form, if applicable,
ii) Maintaining the liquid vehicle at a temperature below the melting point of fenofibrate or a pharmaceutically acceptable salt thereof,
iii) Dissolving the desired amount of fenofibrate in the vehicle,
iv) Spraying the resulting solution onto a solid carrier having a temperature below the melting point of the vehicle,
v) Mechanically working the resulting composition to obtain particles, i.e. a particulate material containing fenofibrate,
   and, prior to or simultaneous with or after applying steps i) to v),
vi) Bringing the vehicle in liquid form, if applicable,
vii) Maintaining the liquid vehicle at a temperature below the melting point of atorvastatin or a pharmaceutically acceptable salt thereof,
viii) Dissolving the desired amount of atorvastatin in the vehicle,
ix) Spraying the resulting solution onto a solid carrier having a temperature below the melting point of the vehicle,
x) Mechanically working the resulting composition to obtain particles, i.e. a particulate material containing atorvastatin, followed by the steps of
xi) Mixing the particulate material containing fenofibrate and the particulate material containing atorvastatin, and
xii) Optionally subjecting the particulate material to conventional methods for preparing solid dosage forms.

In yet another embodiment, the solid oral dosage form of the invention is prepared by a method comprising the steps of:
i) Bringing the vehicle in liquid form, if applicable,
ii) Maintaining the liquid vehicle at a temperature below the melting point of fenofibrate or a pharmaceutically acceptable salt thereof,
iii) Dissolving the desired amount of fenofibrate in the vehicle,
iv) Spraying the resulting solution onto a solid carrier having a temperature below the melting point of the vehicle,
v) Mechanically working the resulting composition to obtain particles, i.e. a particulate material containing fenofibrate,
   and, prior to or simultaneous with or after applying steps i) to v),
vi) Micronizing atorvastatin or a pharmaceutically acceptable salt thereof, if applicable, followed by the steps of
vii) Mixing the particulate material containing fenofibrate and micronized atorvastatin, and
viii) Optionally subjecting the particulate material to conventional methods for preparing solid dosage forms.

In an important embodiment of the invention, at least part of the active substances is present in the composition in the form of a solid dispersion including a molecular dispersion and a solid solution. Normally, about 10% or more such as, e.g., about 20% or more, about 30% or more, about 40% or more, about 50% or more, about 60% or more, about 70% or more, about 80% or more, about 90% or more such as, e.g., about 95% or more or about 100% w/w of either the fibrate or the statin is present in the vehicle in the form of a solid dispersion, provided that at least about 80% w/w of the total amount of active substances is dissolved in the vehicle.

The pharmaceutical compositions comprising the active substance at least partly in form of a solid dispersion or solution may in principle be prepared using any suitable procedure for preparing pharmaceutical compositions known within the art.

A solid dispersion may be obtained in different ways e.g., by employing organic solvents or by dispersing or dissolving the active substance in another suitable medium (e.g. an oily material that is in liquid form at room temperature or at elevated temperatures). Solid dispersions (solvent method) are prepared by dissolving a physical mixture of the active substance (e.g. a drug substance) and the carrier in a common organic solvent, followed by evaporation of the solvent. The carrier is often a hydrophilic polymer. Suitable organic solvents include pharmaceutical acceptable solvent in which the active substance is soluble such as methanol, ethanol, methylene chloride, chloroform, ethylacetate, acetone or mixtures thereof.

Suitable water-soluble carriers include polymers such as polyethylene glycol, poloxamers, polyoxyethylene stearates, poly-epsilon-caprolactone, polyvinylpyrrolidone (PVP), polyvinylpyrrolidone-polyvinylacetate copolymer PVP-PVA (Kollidon VA64), poly-methacrylic polymers (Eudragit RS, Eudragit RL, Eudragit NE, Eudragit E) and polyvinyl alcohol (PVA), hydroxypropyl cellulose (HPC), hydroxypropyl methyl cellulose (HPMC), methyl cellulose, and poly(ethylene oxide) (PEO).

Polymers containing acidic functional groups may be suitable for solid dispersions, which release the active substance in a preferred pH range providing acceptable absorption in the intestines. Such polymers may be one ore more selected from the group comprising hydroxypropyl methylcellulose phtalate (HMPCP), polyvinyl acetate phtalate (PVAP), hydroxypropylmethylcellulose acetate succinate (HPMCAS), alginate, carbomer, carboxymethylcellulose, methacrylic acid copolymer (Eudragit L, Eudragit S), shellac, cellulose acetate phthalate (CAP), starch glycolate, polacrylin, methyl cellulose acetate phtalate, hydroxypropyulcellulose acetate phthalate, cellulose acetate terephtahalate, cellulose acetate isophthalate and cellulose acetate trimellitate.

The weight ratio of active substance to polymer may be in a range of from about 3:1 to about 1:20. However, narrower ranges of from about 3:1 to about 1:5, such as, e.g., from about 1:1 to about 1:3 or about may also be used.

Apart from using the organic solvent based method, solid dispersion or solid solutions of one or more fibrates may be also obtained by dispersing and/or dissolving the active compound in the carrier composition used in the controlled agglomeration method. Stabilizing agents etc. may be added in order to ensure the stability of the solid dispersion/solution.

Solid Dosage Forms

The pharmaceutical composition according to the invention is in particulate form and may be employed as such. However, in many cases it is more convenient to present the composition in the form of granules, pellets, microspheres, nanoparticles and the like or in the form of solid dosage forms including tablets, tablets, beads, capsules, grains, pills, granulates, granules, powder, pellets, sachets, lozenges, troches and the like.

A solid dosage form according to the invention may be a single unit dosage form or it may in the form of a polydepot dosage form contain a multiplicity of individual units such as, e.g., pellets, beads and/or granules.

Usually, a pharmaceutical composition or a solid dosage form of the invention is intended for administration via the oral, buccal or sublingual administration route.

The invention also relates to the above-mentioned presentation form. Within the scope of the invention are compositions/solid dosage forms that are intended to release the active substance in a fast release, a delayed release or modified release manner.

A solid dosage form according to the present invention comprises a pharmaceutical composition in particulate form as described above. The details and particulars disclosed under this main aspect of the invention apply mutatis mutandis to the other aspects of the invention. Accordingly, the properties with respect to increase in bioavailability, changes in bioavailability parameters, reduction in adverse food effect as well as release of one or more fibrates etc. described and/or claimed herein for pharmaceutical compositions in particulate form are analogues for a solid dosage form according to the present invention.

The solid dosage form of the invention, i.e. in unit dosage form, comprises comprises from about 130 to about 170 mg of fenofibrate and from about 5 to about 80 mg of atorvastatin or a pharmaceutically acceptable salt thereof. In a preferred embodiment, the unit dosage form comprises about 160 mg of fenofibrate, or about 145 mg of fenofibrate, and about 10 mg of atorvastatin, or about 15 mg of atorvastatin, or about 20 mg of atorvastatin, or about 25 mg of atorvastatin, or about 30 mg of atorvastatin, or about 40 mg of atorvastatin, or of a pharmaceutically acceptable salt of atorvastatin. Preferably, the unit dosage form comprises fenofibrate and atorvastatin or pharmaceutically acceptable salt thereof in the (relative) weight ratio between fenofibrate and atorvastatin or a pharmaceutically acceptable salt thereof from about 1:1 to about 40:1.

Usually, the concentration of the pharmaceutical composition in particulate form is in a range of from about 5 to 100% w/w such as, e.g., from about 10% to about 90% w/w, from about 15% to about 85% w/w, from about 20% to about 80% w/w, from about 25% to about 80% w/w, from about 30% to about 80% w/w, from about 35% to about 80% w/w, from about 40% to about 75% w/w, from about 45% to about 75% w/w or from about 50% to about 70% w/w of the dosage form. In an embodiment of the invention, the concentration of the pharmaceutical composition in particulate form is 50% w/w or more of the dosage form.

The solid dosage forms of the invention are very stable. For example, the fibrate is present in an amount of at least about 90%, or at least about 95%, or at least about 100%, relative to the amount prior to storage, when assayed after 3 months of storage at a temperature of about 40° C. and a relative humidity of about 75%. Also, the physical stability is very high as can be seen from the Examples below.

The solid dosage form according to the invention is obtained by processing the particulate material according to the invention by means of techniques well-known to a person skilled in the art. Usually, this involves further addition of one or more of the pharmaceutically acceptable excipients mentioned herein.

The composition or solid dosage form according to the invention may be designed to release fenofibrate and/or atorvastatin in any suitable manner provided that the increase in bioavailability is maintained. Thus, the active substance(s) may be released relatively fast in order to obtain an enhanced on-set of action, it may be released so as to follow zero or first order kinetics or it may be released in a controlled or modified manner in order to obtain a predetermined pattern of release. Plain formulations are also within the scope of the present invention.

The composition or solid dosage form according to the invention may also be coated with a film coating, an enteric coating, a modified release coating, a protective coating, an anti-adhesive coating etc.

A solid dosage form according to the invention may also be coated in order to obtain suitable properties e.g. with respect to release of the active substance. The coating may be applied on single unit dosage forms (e.g. tablets, capsules) or it may be applied on a polydepot dosage form or on its individual units.

Suitable coating materials are e.g. methylcellulose, hydroxypropylmethylcellulose, hydroxypropylcellulose, acrylic polymers, ethylcellulose, cellulose acetate phthalate, polyvinyl acetate phthalate, hydroxypropyl methylcellulose phthalate, polyvinylalcohol, sodium carboxymethylcellulose, cellulose acetate, cellulose acetate phthalate, gelatin, methacrylic acid copolymer, polyethylene glycol, shellac, sucrose, titanium dioxide, carnauba wax, microcrystalline wax, zein.

Plasticizers and other ingredients may be added in the coating material. The same or different active substance may also be added in the coating material.

The pharmaceutical composition or a solid dosage form according to the invention is designed to release the fibrate in a suitable manner. Specific release patterns are disclosed in the appended claims to which reference is made. Herein is also given specific relevant absorption patterns. In specific embodiments, the compositions (i.e. particulate material or the solid dosage form) may increase the bioavailability of the fibrate and/or the statin after oral administration. The active substances may be released relatively fast in order to obtain an enhanced on-set of action, it may be released so as to follow zero or first order kinetics or it may be released in a controlled or modified manner in order to obtain a predetermined pattern of release. Plain formulations are also within the scope of the present invention.

In a specific embodiment a solid dosage form of the invention results in an increased bioavailability of fenofibrate and/or atorvastatin relative to existing commercial fenofibrate and/or atorvastatin dosage forms when administered to a mammal in need thereof.

With respect to fenofibrate a solid dosage form according to the invention may provide an AUC₀₋₂₄ value of fibric acid relative to that of commercially available TRICOR® tablets of at least about 1.1, or at least about 1.2, or at least about 1.3, or at least about 1.4, or at least about 1.5, or at least about 1.75 or more, or at least about 2.0, or at least about 2.5, or at least about 3.0, the AUC₀₋₂₄ values being determined under similar conditions. Moreover, a solid dosage form may provide a cₘₐₓ value relative to that of commercially available TRICOR® tablets of at least about 1.1, or at least about 1.2, or at least about 1.3, or at least about 1.4, or at least about 1.5, or at least about 1.6 or more, or at least about 2.0, or at least about 2.5, or at least about 3.0, the cₘₐₓ values being determined under similar conditions.

With respect to atorvastatin, a solid dosage form according to the invention may provide an AUC₀₋₂₄ value relative to that of commercially available LIPITOR™ tablets of at least about 1.0, or at least about 1.1, or at least about 1.2, or at least about 1.3, or at least about 1.4, or at least about 1.75 or more, or at least about 2.0, or at least about 2.5, or at least about 3.0, the AUC₀₋₂₄ values being determined under similar conditions. Moreover, a solid dosage form may provide a cₘₐₓ value relative to that of commercially available LIPITOR™ tablets of at least about 1.0, or at least about 1.1, or at least about 1.2, or at least about 1.3, or at least about 1.4, or at least about 1.5 or more, or at least about 2.0, or at least about 2.5, or at least about 3.0, the cₘₐₓ values being determined under similar conditions.

In a preferred embodiment, the present invention provides a combination solid dosage form comprising fenofibrate and atorvastatin, which dosage form provides an AUC₀₋₂₄value of fenofibrate (fibric acid) relative to that of atorvastatin when measured in humans (human blood plasma) of at least 250, or at least about 500, or at least about 980, or at least about 2000, the AUC₀₋₂₄ values being determined under similar conditions and using the same plasma or identical samples.

In another preferred embodiment, the present invention provides a combination solid dosage form comprising fenofibrate and atorvastatin, which dosage form provides an AUC₀₋₂₄value of fenofibrate (fibric acid) relative to that of atorvastatin when measured in humans (human blood plasma) of less than about 10,000, or less than about 5100, or less than about 4000, or less than about 2100, the AUC₀₋₂₄ values being determined under similar conditions and using the same or identical plasma samples.

In a typical average blood plasma sample, the AUC₀₋₂₄ of fenofibrate resulting from the administration of 160 mg fenofibrate tablets are about 118,300 h·ng/mL. However, wide individual variations in bioavailability are usually observed.

Other Aspects of the Invention

A pharmaceutical composition or a solid dosage form according to the invention is designed to release the fibrate in a suitable manner. Specific release patterns as well as specific absorption patterns are mentioned below.

In specific embodiments, the fibrate and/or the statin is released from the composition within about 2 hours such as, e.g., within about 1.5 hours or within about 1 hour after oral administration, and/or about 50% w/w or more of the fibrate and/or the statin is released from the composition within about 30 min after oral administration, and/or about 50% w/w or more of the fibrate and/or the statin is released from the composition within about 20 min after oral administration, and/or about 60% w/w or more of the fibrate is released from the composition within about 1.5 hours after oral administration, and/or about 60% w/w or more of the fibrate and/or the statin is released from the composition within about 1 hour after oral administration, and/or about 70% w/w or more of the fibrate and/or the statin is released from the composition within about 1.5 hours after oral administration, and/or about 70% w/w or more of the fibrate and/or the statin is released from the composition within about 1 hour after oral administration, and/or about 85% w/w or more of the fibrate and/or the statin is released from the composition within about 45 min when tested in an in vitro dissolution test according to USP dissolution test (paddle) employing water as dissolution medium, 100 rpm and a temperature of about 37° C.

In another embodiment about 50% w/w or more of the fibrate and/or the statin is released from the composition within about 20 min, 15 min or 10 min, and/or about 60% w/w or more of the fibrate and/or the statin is released from the composition within about 20 min or 15 min, and/or about 70% w/w or more of the fibrate and/or the statin is released from the composition within about 20 min or 15 min, when tested in an in vitro dissolution test according to USP dissolution test (paddle) employing water as dissolution medium, 100 rpm and a temperature of about 37° C.

In a still further embodiment about 50% w/w or more of the fibrate and/or the statin contained in the composition is absorbed within about 8 hours, 7 hours, 6 hours or 5 hours, and/or about 60% w/w or more of the fibrate and/or statin contained in the composition is absorbed within about 8 hours or 7 hours after oral administration, and/or about 60% w/w or more of the fibrate contained in the composition is absorbed within about 7 hours after oral administration, and/or about 70% w/w or more of the fibrate contained in the composition is absorbed within about 8 hours or 7 hours after oral administration.

The details and particulars disclosed under this main aspect of the invention apply mutatis mutandis to the other aspects of the invention. Accordingly, the properties with respect to increase in bioavailability, changes in bioavailability parameters, reduction in adverse food effect as well as release of one or more fibrates etc. described and/or claimed herein for pharmaceutical compositions in particulate form are analogues for a solid dosage form according to the present invention.

Materials and Methods

Materials

Fenofibrate (supplied by Sigma)
Lactose monohydrate 200 mesh (from DMV)
Granulated silicium oxide, AEROPERL® 300, (Degussa)
Polyethylene glycol 6000, PLURACOL® E6000 (from BASF)
Poloxamer 188, PLURONIC® F-68 (from BASF)
Glyceryl monostearate, RYLOO MD50, (from Danisco Cultor), Ph.Eur.
Avicel PH200 (microcrystalline cellulose) (from FMC)
Magnesium stearate

Tablets, capsules or granules might be enteric coated with different types of polymers such as hydroxypropylmethylcellulose acetate succinate (Aqoat), cellulose acetate phthalate CAP, hydroxypropylmethylcellulose phtalate HPMCP or methacrylic acid copolymers such as Eudragit L30D, Eudragit 100/S, Eudragit 100/L.

Tricor Tablet Formulation

TRICOR® tablets from Abbott Laboratories are fenofibrate-containing tablets available for oral administration, either containing 54 mg or 160 mg of fenofibrate per tablet.

The tablets contain the following inactive ingredients: colloidal silicon dioxide, crospovidone, lactose monohydrate, lecithin, microcrystalline cellulose, polyvinyl alcohol, povidone, sodium lauryl sulfate, sodium stearyl fumarate, talc, titanium dioxide, xanthan gum, colorant.

Equipment

Laboratory scale fluid bed equipment: Strea-1.

The melt feed unit is a prototype composed of separate units for heating of air supplies for the atomizer, pressure tank and feeding tube. Granulate was sieved manually and mixed with extragranular excipients in a Turbula mixer.

Tablet compression was performed on a single punch press, Diaf TM20

Methods

According to the method of the invention, the fenofibrate drug was dissolved into the melted vehicle(s) and applied on the particulate carrier(s) as follows:

The vehicle(s) was melted in a beaker placed in a microwave oven. The beaker was transferred to a temperature controlled heating plate supplied with magnetic stirring. Fenofibrate was dissolved slowly in the melt at a temperature of 75° C. under magnetic stirring. The hot solution was transferred to the pressure tank for melt spray application onto the carrier in the fluid bed. The granulate product was discharged from the fluid bed and sieved through sieve 0.7 mm or 1.0 mm manually. The sieved product was blended with magnesium stearate for 0.5 min in a Turbula mixer. If an extragranular phase has to be incorporated, the extragranular phase was premixed with the granulate in 3 minutes in a Turbula mixer.

The tablet compression was performed on a single punch machine Diaf TM20.

Threshold Test

The test involves determination of flowability according to the method described in Ph.Eur. by measuring the flow rate of the material out of a funnel with a nozzle diameter of 10.0 mm.

Viscoleo (medium chain triglycerides MCT; Miglyol 812 N from Condea) was added to 100 g of the solid pharmaceutically acceptable material to be tested for use according to the invention and mixed manually. The mixture obtained was sieved through sieve 0.3 mm to assure a homogenous mixture. The oil was added successively until a flow of 100 g of the mixture could not flow through the nozzle. If the material to be tested has a high bulk volume (e.g. like that of Aeroperl 300) only 50 g of the mixture is used when testing these blends. The maximal concentration of oil where flow of material could be obtained is called the Threshold Value (given as % w/w).

Release Test

A fat-soluble colorant Sudan II (BDH GUR®) obtained from BDH VWR International 14.3 mg was dissolved in 50.0 g viscoleo (fractionated medium chain triglycerides).

10 g of the oil was added to 10.0 g of the solid pharmaceutically acceptable material to be tested for use according to the present invention and mixed until the oil was fully absorbed in the solid material. The mixture was subsequently sieved through sieve 0.3 mm to achieve a homogeneous mixture.

1.00 g of the mixture was transferred to a centrifugal tube and 3.00 ml of water was added. The suspension was mixed in a blood sample turner for 1 hour and subsequently centrifuged for 10 minutes at 5000 rpm. The upper phase of oil and water was transferred carefully to a beaker and the water was evaporated in an oven at 80° C. until constant weight. The amount of oil released from the solid material was calculated on basis of the weight of the remaining after evaporation of the water phase.

Disintegration Test

The disintegration time was determined according to the method described in to Ph. Eur.

Dissolution Test

The test was performed in accordance with Ph. Eur 2.9.3 using the paddle apparatus. The quantification was performed using HPLC with UV-detection.

| | |
|---|---|
| Medium: | 900 ml water with 0.75% sodium |
| | lauryl sulfate (SLS) |
| Rotation speed: | 50 rpm |
| Temperature: | 37° C. |
| Sampling time: | 10, 20, 30, 45 and 60 minutes |
| Acceptance criteria: | >75% at 45 minutes (for the stability study) |

Determination of Bulk Density

The bulk density was measured by pouring 100 g of the powder in question in a 250 ml graduated cylinder. The bulk density is given as the tapped bulk density in g/ml. The determination was performed according to Ph. Eur. (apparent volume).

Determination of Oil Absorption Value

The oil absorption value is determined by adding well-defined amounts (a 10 g) of viscoleo to a well-defined amount of the pharmaceutically acceptable material (100 g) to be tested. The oil absorption value (expressed as g viscoleo/100 g material) is reached when a further addition of 10 g oil results in a material that does not have suitable properties with respect to flowability, i.e. the material does not meet the meet the requirements when tested according to Ph.Eur. (flowability test; see above under Threshold Test herein).

Determination of BET Surface Area

The apparatus applied was a Micromertics Gemini 2375. The method applied was according to USP volumetric methods based on multiple point determination.

Determination of Flowability

The flowability was determined according to the method described in Ph.Eur. measuring the flow rate of the material out of a funnel with a nozzle diameter of 10.0 mm.

Determination of Weight Variation

The tablets prepared in the Examples herein were subject to a test for weight variation performed in accordance with Ph. Eur.

Determination of Average Tablet Hardness

The tablets prepared in the Examples herein were subject to at test for tablet hardness employing Schleuniger Model 6D apparatus and performed in accordance with the general instructions for the apparatus.

Determination of Solid Solution

According to the present invention, the fibrate is dissolved in a vehicle. In order to substantiate this, a test involving differential scanning calometry is performed. The test is performed on the particulate composition, solid dosage form or mixture of vehicle and fibrate (after the solid solution is supposed to form). Standard DSC equipment connected to a PC is used.

| | |
|---|---|
| Sample size: | 10 mg in alu pans |
| Heating rate: | 5° C./min from 27° C. to 110° C. |
| Evaluation: | The fibrate and statin are considered to be in |
| | dissolved state or non-crystalline if neither |
| | fibrate nor statin endoterm peaks are observed |
| | and if the melting intervals do not significantly |
| | shift compared with the vehicle alone. |

Determination of Geometric Weight Mean Diameter d_{gw}

The geometric weight mean diameter was determined by employment of a method of laser diffraction dispersing the particulate material obtained (or the starting material) in air. The measurements were performed at 1 bar dispersive pressure in Sympatec Helos equipment, which records the distribution of the equivalent spherical diameter. This distribution is fitted to a log normal volume-size distribution.

When used herein, “geometric weight mean diameter” means the mean diameter of the log normal volume-size distribution.

In vivo Studies in Beagle Dogs

In vivo studies with the purpose of determining the bioavailability of the compositions of the present invention relative to the bioavailability of the commercially available fenofibrate tablet formulation, i.e. TRICOR®, was performed using Beagle dogs.

The experimental work was performed in Denmark using four male Beagle dogs each having a body weight of 12-18 kg (starting weight). The studies were conducted as open, non-randomised, cross-over studies. Each animal was its own control. Oral doses of fenofibrate was administered according to the data below.

The dogs were fasted overnight prior to dosing (water ad libitum) and were fed 5 hours after dosing (water ad libitum). Each dog was dosed with the specified dose of fenofibrate without taking the weight of the dog into consideration.

Blood samples were collected at vena jugularis externa at the following points of time: Pre-dose, 1, 1.5, 2, 3, 4, 6, 8, 12 and 24 hours after dosing. 4 ml of blood were collected, mixed with EDTA, and the samples were frozen (−80° C.). The blood samples were analyzed using on-line extraction LC/MS and results were given in mg/mL.

The determined full blood concentration profiles of fenofibrate were treated using the Pharmacokinetic software WINNONLIN®, (Pharsight, Calif.; USA) to calculate the pharmacokinetic parameters. All data are dose adjusted, when necessary.

The following examples serve the purpose of illustration of the invention and are not intended to limiting the scope of the present invention.

### EXAMPLE 1

Immediate Release Tablet Containing a Fenofibrate and Atorvastatin

| Substance | Ingredient | % | mg |
|---|---|---|---|
| Drug | Fenofibrate | 23.9 | 160.00 |
| Drug | Atorvastatin | 1.5 | 10.00 |
| Carrier | Lactose | 37.6 | 247.64 |
| Vehicle | PEG 6000 | 25.6 | 170.88 |
| Vehicle | Poloxamer 188 | 11.0 | 73.24 |
| Excipient | Magnesium stearate | 0.4 | 2.69 |
| | Total | 100.00 | 667.45 |

Fenofibrate and atorvastatin are mainly dissolved in Polyethylene glycol 6000 and Poloxamer 188 (70:30 w/w ratio) at 70° C. The dispersion is sprayed on 250 g lactose in a fluid bed Phast FB-100 with a Phast FS-1.7 melt-spray unit. The particular material obtained is sieved through sieve 0.7 mm and blended with magnesium stearate for 0.5 min in a Turbula mixer.

The powder mixture is compressed into 13 mm tablets with strength of 160 mg fenofibrate and 10 mg atorvastatin in to a 667 mg tablet with compound cup shaped. Mean disintegration time: 20 min, Hardness: 45 N

### EXAMPLE 2

Immediate Release Tablet Containing Fenofibrate and Atorvastatin

| Substance | Ingredient | % | mg |
|---|---|---|---|
| Drug | Fenofibrate | 23.2 | 160.00 |
| Drug | Atorvastatin | 2.9 | 20.00 |
| Carrier | Lactose | 37.9 | 261.00 |
| Vehicle | PEG 6000 | 24.9 | 171.00 |
| Vehicle | Poloxamer 188 | 10.6 | 73.00 |
| Excipient | Magnesium stearate | 0.5 | 3.00 |
| | Total | 100.00 | 688.00 |

Fenofibrate and atorvastatin are mainly dissolved in Polyethylene glycol 6000 and Poloxamer 188 (70:30 w/w ratio) at 70° C. The dispersion is sprayed on 250 g lactose in a fluid bed Phast FB-100 with a Phast FS-1.7 melt-spray unit. The particular material obtained is sieved through sieve 0.7 mm and blended with magnesium stearate for 0.5 min in a Turbula mixer.

The powder mixture is compressed into 13 mm tablets with strength of 160 mg fenofibrate and 20 mg atorvastatin into a 688 mg tablet with compound cup shaped. Mean disintegration time: 25 min, Hardness: 47 N

### EXAMPLE 3

Immediate Release Tablet Containing Fenofibrate and Atorvastatin

| Substance | Ingredient | % | mg |
|---|---|---|---|
| Drug | Fenofibrate | 24.3 | 160.00 |
| Drug | Atorvastatin | 1.5 | 10.00 |
| Carrier | Lactose | 36.7 | 241.00 |
| Vehicle | PEG 6000 | 26.0 | 171.00 |
| Vehicle | Poloxamer 188 | 11.0 | 73.00 |
| Excipient | Magnesium stearate | 0.5 | 3.00 |
| | Total | 100.00 | 658.00 |

Fenofibrate and Atorvastatin are mainly dissolved in Polyethylene glycol 6000 and Poloxamer 188 (70:30 w/w ratio) at 70° C. The dispersion is sprayed on 250 g lactose in a fluid bed Phast FB-100 with a Phast FS-1.7 melt-spray unit. The particulate material obtained is sieved through sieve 0.7 mm and blended with magnesium stearate for 0.5 min in a Turbula mixer.

The powder mixture is compressed into 12 mm tablets with strength of 160 mg fenofibrate and 10 mg Atorvastatin into a 658 mg tablet with compound cup shaped. Mean disintegration time: 22 min, Hardness: 41 N

### EXAMPLE 4

Immediate Release Tablet Containing Fenofibrate and Atorvastatin

| Substance | Ingredient | % | mg |
|---|---|---|---|
| Drug | Fenofibrate | 23.2 | 160.00 |
| Drug | Atorvastatin | 1.4 | 10.00 |
| Carrier | Lactose | 39.4. | 271.00 |
| Vehicle | PEG 6000 | 24.9 | 171.00 |
| Vehicle | Poloxamer 188 | 10.6 | 73.00 |
| Excipient | Magnesium stearate | 0.5 | 3.00 |
| | Total | 100.00 | 688.00 |

Fenofibrate and Atorvastatin are mainly dissolved in Polyethylene glycol 6000 and Poloxamer 188 (70:30 w/w ratio) at 70° C. The dispersion is sprayed on 250 g lactose in a fluid bed Phast FB-100 with a Phast FS-1.7 melt-spray unit. The particulate material is sieved through sieve 0.7 mm and blended with magnesium stearate for 0.5 min in a Turbula mixer.

The powder mixture is compressed into 13 mm tablets with strength of 160 mg fenofibrate and 10 mg atorvastatin into a 688 mg tablet with compound cup shaped. Mean disintegration time: 25 min, Hardness: 39 N

### EXAMPLE 5

Tablet Based on Lipophilic Matrix of Glyceryl Monostearate

| Substance | Ingredient | % | mg |
|---|---|---|---|
| Drug | Fenofibrate | 28.0 | 160.00 |
| Drug | Atorvastatin | 1.7 | 10.00 |
| Carrier | Lactose 200 mesh | 17.5 | 100.00 |
| Vehicle | Glycerylmonostearate | 52.4 | 300.00 |
| Excipient | Magnesium stearate | 0.4 | 2.00 |
| | | 100.0 | 572.00 |

Fenofibrate and Atorvastatin are mainly dissolved in Glyceryl monostearate at 70° C. The solution is sprayed on 200 g lactose in a fluid bed Phast FB-100 with a Phast FS-1.7 melt-spray unit. The particulate material is sieved through sieve 0.7 mm and blended with magnesium stearate for 0.5 min in a Turbula mixer.

The powder mixture is compressed into 11 mm tablets with 572 mg tablet with compound cup shape.

Mean disintegration time: 45 min, Hardness: 48 N

### EXAMPLE 6

Modified Release Polydepot Capsule based on Swelling Hydrocolloid Matrix of Hydroxypropylcellulose

| Substance | Ingredient | % | mg |
|---|---|---|---|
| Drug | Fenofibrate | 23.5 | 160.00 |
| Drug | Atorvastatin | 2.9 | 20.00 |
| Carrier | HPMC 2910 3 cp | 22.1 | 150.00 |
| Carrier | Lactose 200 mesh | 7.4 | 50.00 |
| Vehicle | Glyceryl monostearate | 44.1 | 300.00 |
| | Total | 100.00 | 680.00 |

Fenofibrate and Atorvastatin are mainly dissolved in Glycerylmonostearate at 70° C. The solution is sprayed on a mixture of 50 g lactose and 150 g HPMC in a fluid bed Phast FB-100 with a Phast FS-1.7 melt-spray unit. The particulate material is sieved through sieve 0.7 mm and filled into hard gelatine capsules (680 mg)

### EXAMPLE 7

Immediate Release Tablet

| Substance | Ingredient | % | mg |
|---|---|---|---|
| Drug | Fenofibrate | 33.5 | 160.00 |
| Drug | Atorvastatin | 4.2 | 20.00 |
| Oil-sorption material | Aeroperl 300 | 19.9 | 95.00 |
| Vehicle | PEG 3000 | 41.8 | 200.00 |
| Excipient | Magnesium stearate | 0.6 | 3.00 |
| | Total | 100.00 | 478.00 |

Fenofibrate and Atorvastatin are mainly dissolved in Polyethylene glycol 3000 at 70° C. The dispersion is sprayed on 2509 Aeroperl in a fluid bed Phast FB-100 with a Phast FS-1.7 melt-spray unit. The particulate material is sieved through sieve 0.7 mm and blended with magnesium stearate for 0.5 min in a Turbula mixer.

The powder mixture is compressed into 11 mm tablets with strength of 160 mg fenofibrate and 20 mg atorvastatin into a 478 mg tablet with compound cup shaped. Mean disintegration time: 29 min, Hardness: 51 N

### EXAMPLE 8

Solid Dosage Forms According to the Invention

The following compositions were prepared according to the method described in Example 1 above.

| Sub- | | A | B | C | D | E |
|---|---|---|---|---|---|---|
| stance | Ingredient | mg (%) | mg (%) | mg (%) | mg (%) | mg (%) |
| Drug | Fenofibrate | 160.09 | 50.05 | 50.08 | 50.09 | 159.99 |
| Drug | Atorvastatin | 10.0 | 20.0 | 30.0 | 40.0 | 50.0 |
| Vehicle | PEG6000 | 208.12 | 171.09 | 124.29 | — | — |
| 1 | PEG4000 | — | — | — | 244.57 | — |
| | GMS | — | — | — | — | 86.15 |
| Vehicle | Poloxamer188 | 89.19 | 73.33 | 53.27 | — | — |
| 2 | | | | | | |
| Carrier | Lactose | 356.51 | 231.87 | — | 232.02 | 163.01 |
| | Aeropearl | — | — | 63.89 | — | — |
| | 300 | | | | | |
| Excipi- | Mg stearate | 4.09 | 2.65 | 1.47 | 5.32 | 8.35 |
| ents | Avicel | — | — | — | — | 417.50 |

### EXAMPLE 9

Preferred Ranges of Fenofibrate and Atorvastatin in a Composition According to the Invention

Compositions e.g. as described in Examples 1-7 can be varied in order to adjust the contained amount of the fibrate and the statin. A person skilled in the art will know how to adjust the amount of active substances and the pharmaceutically acceptable excipients without departing from the inventive object. In the following is given suitable ranges for fenofibrate and individual statins in compositions of the invention: Fenofibrate 140-170 mg in combination with atorvastatin 10-80 mg, for example 160 mg fenofibrate and 10 mg atorvastatin, or 145 mg fenofibrate and 10 mg atorvastatin.

### EXAMPLE 10

Stability of Compositions According to the Invention

For drug substances like fenofibrate and statins moisture is a significant threat to the stability of the compounds. This is especially true when one tries to formulate two unstable compounds into on single tablet unit. Very small amounts of moisture/water can significantly increase the “drug interaction” degradation. Also crystal growth is a potential threat for moisture containing combination products.

By the uniqueness of the formulation, the avoidance of water in the process, and the careful selection of low water containing ingredients, excellent stability of the compounds is ensured. On an average the total water content of the final formulation is below 0.5% w/w. The polymer matrix serves as a moisture/oxygen protective cover of the labile molecules.

### EXAMPLE 11 (A-E)

Methods of Manufacturing Fenofibrate—Atorvastatin Combinations

There are several useful methods for preparing combination products according to this invention. The methods is primarily selected from the desired characteristics and performance of the composition or solid dosage form. In examples 11A-11E is given a number of compositions and methods of production. The methods shown are by no means intended to limit the scope of this invention.

All granulates listed herein can either be filled into hard gelatin capsules or compressed into tablets.

The following fenofibrate granulate is disclosed in from international application PCT/DK2004/000667:

| Substance | Ingredient | % | mg |
|---|---|---|---|
| Drug | Fenofibrate | 19.6 | 160.00 |
| Carrier | Lactose | 43.6 | 356.50 |
| Vehicle | PEG 6000 | 25.4 | 208.20 |
| Vehicle | Poloxamer 188 | 10.9 | 89.20 |
| Excipient | Magnesium stearate | 0.5 | 4.10 |
| | | 100.0 | 818.00 |

### EXAMPLE 11A

The fenofibrate granulate according to international application no. PCT/DK2004/000667 was used.

The fenofibrate granulate is mixed with another granulate containing atorvastatin. This statin granulate is as follows:

| Substance | Ingredient | % | Mg |
|---|---|---|---|
| Drug | Atorvastatin | 6.7 | 10.00 |
| Carrier | Lactose 200 mesh | 33.3 | 50.00 |
| Vehicle | PEG 6000 | 44.0 | 66.00 |
| Vehicle | Poloxamer 188 | 14.7 | 22.00 |
| Excipient | Magnesium stearate | 1.3 | 2.00 |
| | | 100.0 | 150.00 |

The granulate obtained is sieved through sieve 0.7 mm and blended with the fenofibrate granulate and magnesium stearate for 0.5 min in a Turbula mixer.

The final granulate is compressed into 13.5 mm tablets with strength of 160 mg fenofibrate and 10 mg Atorvastatin into a 970 mg tablet with compound cup shaped. Mean disintegration time: 24 min, Hardness: 49 N

### EXAMPLE 11B

A single granulate comprising fenofibrate and atorvastatin is made as follows:

| Substance | Ingredient | % | mg |
|---|---|---|---|
| Drug | Fenofibrate | 19.5 | 160.00 |
| Drug | Atorvastatin | 1.2 | 10.00 |
| Carrier | Lactose | 42.6 | 349.00 |
| Vehicle | PEG 6000 | 25.4 | 208.00 |
| Vehicle | Poloxamer 188 | 10.8 | 89.00 |
| Excipient | Magnesium stearate | 0.5 | 4.00 |
| | Total | 100.00 | 820.00 |

Fenofibrate and Atorvastatin are mainly dissolved in polyethylene glycol 6000 and Poloxamer 188 (70:30 w/w ratios) at 70° C. The dispersion is sprayed on 250 g lactose in a fluid bed Phast FB-100 with a Phast FS-1.7 melt-spray unit. The particulate material obtained is sieved through sieve 0.7 mm and blended with magnesium stearate for 0.5 min in a Turbula mixer.

The granulate is compressed into 13.5 mm tablets with strength of 160 mg fenofibrate and 10 mg Atorvastatin into a 820 mg tablet with compound cup shaped. Mean disintegration time: 21 min, Hardness: 53 N

### EXAMPLE 11C

A single granulate comprising fenofibrate and atorvastatin is made as follows:

| Substance | Ingredient | % | mg |
|---|---|---|---|
| Drug | Fenofibrate | 19.5 | 160.00 |
| Drug | Atorvastatin | 1.2 | 10.00 |
| Carrier | Lactose | 42.6 | 349.00 |
| Vehicle | PEG 6000 | 25.4 | 208.00 |
| Vehicle | Poloxamer 188 | 10.8 | 89.00 |
| Excipient | Magnesium stearate | 0.5 | 4.00 |
| | Total | 100.00 | 820.00 |

Fenofibrate are dissolved in polyethylene glycol 6000 and Poloxamer 188 (70:30 w/w ratios) at 70° C. The dispersion is sprayed on a mixture of 250 g lactose and 7.17 g of atorvastatin in a fluid bed Phast FB-100 with a Phast FS-1.7 melt-spray unit. The particulate material obtained is sieved through sieve 0.7 mm and blended with magnesium stearate for 0.5 min in a Turbula mixer.

The granulate is compressed into 13.5 mm tablets with strength of 160 mg fenofibrate and 10 mg Atorvastatin into a 820 mg tablet with compound cup shaped. Mean disintegration time: 23 min, Hardness: 56 N

### EXAMPLE 11D

A fenofibrate granulate was manufactured according to international application PCT/DK2004/000667.

The fenofibrate granulate is mixed with a granulate similar to the granulate composition of Lipitor™ tablets of either 10, 20 or 40 mg of atorvastatin in order to obtain the same plasma profiles as those of LIPITOR™. LIPITOR™ based granulates may have the following composition(s):

10 mg atorvastatin per 150 mg granulate:

| | | |
|---|---|---|
| Atorvastatin calcium trihydrate | 10.9 | mg |
| Microcrystalline cellulose | 60.0 | mg |
| Calcium carbonate | 33.0 | mg |
| Lactose monohydrate | 32.8 | mg |
| Croscarmellose sodium | 9.0 | mg |
| HPMC | 3.0 | mg |
| Polysorbate 80 | 0.6 | mg |
| Magnesium stearate | 0.7 | mg |

20 mg atorvastatin per 300 mg granulate:

| | | |
|---|---|---|
| Atorvastatin calcium trihydrate | 21.8 | mg |
| Microcrystalline cellulose | 120.0 | mg |
| Calcium carbonate | 66.0 | mg |
| Lactose monohydrate | 65.6 | mg |
| Croscarmellose sodium | 18.0 | mg |
| HPMC | 6.0 | mg |
| Polysorbate 80 | 1.2 | mg |
| Magnesium stearate | 1.4 | mg |

40 mg atorvastatin per 600 mg granulate:

| | | |
|---|---|---|
| Atorvastatin calcium trihydrate | 43.4 | mg |
| Microcrystalline cellulose | 240.0 | mg |
| Calcium carbonate | 132.0 | mg |
| Lactose monohydrate | 131.2 | mg |
| Croscarmellose sodium | 36.0 | mg |
| HPMC | 12.0 | mg |
| Polysorbate 80 | 2.4 | mg |
| Magnesium stearate | 3.0 | mg |

The fenofibrate granulate and the “Pravachol” granulate are mixed in a turbula mixer and the final granulate is then either filled into hard gelatin capsules or compressed into tablet with a suitable crushing strengths around 40-50 N.

### EXAMPLE 11E

A fenofibrate granulate was manufactured according to patent application PCT/DK2004/000667.

The fenofibrate granulate is mixed with micronized atorvastatin, optionally added conventional excipients or additives for tablet production like a glidant, filler, binder, or disintegrator.

The granulate is either filled into hard gelatin capsules or compressed into tablet with a suitable crushing strength.

### EXAMPLE 12

Formulations for In Vivo Studies in Dogs

Compositions of the invention were investigated in in vivo studies in dog. As fenofibrate is a drug substance that has major bioavailability problems, the study was primarily to investigate whether an improved bioavailability could be obtained. Accordingly, no data with respect to the statin component is available.

Tablets of 50 mg and 160 mg strength with respect to fenofibrate, respectively and having the following compositions were prepared as described in Example 1:

| Sub- | | A | B | C | D | E |
|---|---|---|---|---|---|---|
| stance | Ingredient | mg | mg | mg | mg | mg |
| Drug | Feno- | 160.09 | 50.05 | 50.08 | 50.09 | 159.99 |
| | fibrate | | | | | |
| Vehicle | PEG6000 | 208.12 | 171.09 | 124.29 | — | — |
| 1 | PEG4000 | — | — | — | 244.57 | — |
| | GMS | — | — | — | — | 86.15 |
| | (Rylo) | | | | | |
| Vehicle | Polox- | 89.19 | 73.33 | 53.27 | — | — |
| 2 | amer188 | | | | | |
| Carrier | Lactose | 356.51 | 231.87 | — | 232.02 | 163.01 |
| | Aeropearl | — | — | 63.89 | — | — |
| | 300 | | | | | |
| Excipi- | Mg | 4.09 | 2.65 | 1.47 | 5.32 | 8.35 |
| ents | stearate | | | | | |
| | Avicel | — | — | — | — | 417.50 |
| Total | | 818.00 | 529.00 | 293.00 | 532.00 | 835.00 |
| Hardness | N | 60 | 44 | 44 | 47 | 102 |
| Disinte- | Minutes | 25 | 14 | 30 | 48 | >55 |
| gration | | | | | | |
| time | | | | | | |
| Diameter | Mm | Oblong | 12 | 12 | 10 | Oblong |

### EXAMPLE 13

Dissolution Tests

The tablet formulation A from Example 10 was subjected to a dissolution test as described in Methods with the following results:

| Time (min) | % dissolved |
|---|---|
| 0 | 0 |
| 10 | 28 |
| 20 | 56 |
| 30 | 74 |
| 45 | 88 |
| 60 | 97 |

### EXAMPLE 14

Stability Tests

Samples of the tablet formulation A from Example 10 was stored in PP bottles under the following conditions, respectively, and subjected to a dissolution (stability) test as described in Methods after 1 months and 3 months of storage; % dissolved is the percentage of fenofibrate dissolved after 45 minutes:

| | % dissolved | | |
|---|---|---|---|
| | 25° C. and | 30° C. and | 40° C. and |
| Months | 60% RH | 65% RH | 75% RH |
| 0 | 88 | — | — |
| 1 | 99 | 88 | 90 |
| 3 | 90 | 97 | 90 |

Samples of the tablet formulation A was stored under the following conditions, respectively, and subjected to a fibrate assay with the following results:

| | mg fenofibrate | | |
|---|---|---|---|
| | 25° C. and | 30° C. and | 40° C. and |
| Months | 60% RH | 65% RH | 75% RH |
| 0 | 163.8 | — | — |
| 1 | 161.9 | 160.1 | 160.8 |
| 3 | 162.6 | 164.9 | 164.4 |

Samples of the inventive tablet formulation A was stored under the following conditions, respectively, and subjected to a degradation product test according to Ph. Eur. (Degradation products A, B, G and Unknown accumulated into Total Degradation Product; HPLC method) with the following results:

| | Total Degradation Product, % w/w, impurity | | |
|---|---|---|---|
| | 25° C. and | 30° C. and | 40° C. and |
| Months | 60% RH | 65% RH | 75% RH |
| 0 | 0.05 | — | — |
| 1 | 0.05 | 0.05 | 0.05 |
| 3 | 0.05 | 0.05 | 0.05 |

### EXAMPLE 15

In Vivo Study in Dogs

An in vivo study of formulation A from Example 10 160 mg in Beagle dogs, performed as described above under Methods, relative to TRICOR®, 160 mg (Batch no.: 098212E21), gave the following results:

Blood concentrations (mg/mL) (average of 4 dogs) after administration of formulation:

Formulation

| Time | TRICOR ® | Invention, A |
|---|---|---|
| (hr) | (160 mg) | (160 mg) |
| 0 | n.a. | n.a. |
| 0.5 | 367.5 | 995.8 |
| 1.0 | 612.5 | 2209.3 |
| 1.5 | 722.0 | 2627.8 |
| 2.0 | 725.8 | 2097.3 |
| 3.0 | 443.8 | 1219.5 |
| 4.0 | 295.3 | 930.5 |
| 6.0 | 160.5 | 642.0 |
| 8.0 | 250.3 | 869.5 |
| 12.0 | 211.8 | 615.3 |
| 24.0 | 133.3 | 394.0 |
| 48.0 | n.a. | 164.5 |

Relative bioavailability based on AUC (invention, A/TRICOR®): 306%.
Relative cₘₐₓ(invention, A/TRICOR®): 356%.

### EXAMPLE 16

In Vivo Study in Dogs

A second in vivo study of formulation A (Example 10), 160 mg in Beagle dogs, performed as described above under Methods, relative to TRICOR®, 160 mg (Batch no.: 098212E21), gave the following results:

Blood concentrations (mg/mL) (average of 4 dogs) after administration of formulation:

Formulation

| Time | TRICOR ® | Invention, A |
|---|---|---|
| (hr) | (160 mg) | (160 mg) |
| 0 | 0 | 0 |
| 0.5 | 339.3 | 3616.0 |
| 1.0 | 1318.8 | 3724.8 |
| 1.5 | 1313.3 | 2982.0 |
| 2.0 | 1390.0 | 2355.8 |
| 3.0 | 1361.3 | 1359.5 |
| 4.0 | 1019.3 | 1309.5 |
| 6.0 | 969.3 | 973.8 |
| 8.0 | 667.0 | 1113.0 |
| 12.0 | 390.3 | 768.5 |
| 24.0 | 183.3 | 295.0 |
| 48.0 | 85.0 | 302.0 |

Relative bioavailability based on AUC (invention, A/TRICOR®): 198%.
Relative cₘₐₓ(invention, A/TRICOR®): 238%.

### EXAMPLE 17

In Vivo Study in Dogs

An in vivo study of the formulations B, C and D (Example 10), 2×50 mg in Beagle dogs, performed as described above under Methods, relative to LIPANTHYL®67M, 2×67 mg (Batch no.: 75641), gave the following results:

Blood concentrations (mg/mL) (average of 4 dogs) after administration of formulation:

| | Formulation | | | |
|---|---|---|---|---|
| Time | LIPANTHYL ®67M | Invention, B | Invention, C | Invention, D |
| (hr) | (2 × 67 mg) | (2 × 50 mg) | (2 × 50 mg) | (2 × 50 mg) |
| 0 | 0 | 0 | 0 | 0 |
| 0.5 | 187.3 | 2769.5 | 227.3 | 546.0 |
| 1.0 | 669.5 | 3526.8 | 521.5 | 1381.5 |
| 1.5 | 960.3 | 3106.3 | 858.3 | 1615.5 |
| 2.0 | 895.3 | 2938.0 | 989.3 | 1566.8 |
| 3.0 | 433.0 | 2465.5 | 902.5 | 1503.3 |
| 4.0 | 240.0 | 1492.3 | 783.8 | 1719.0 |
| 6.0 | 77.8 | 809.5 | 655.8 | 1034.5 |
| 8.0 | 79.3 | 1202.8 | 409.0 | 1056.0 |
| 12.0 | 291.3 | 848.0 | 269.8 | 597.3 |
| 24.0 | 82.5 | 378.0 | 163.8 | 282.8 |
| 48.0 | 19.3 | 18.8 | 51.5 | 36.5 |
| 72.0 | 0 | 0 | 0 | 0 |

Relative bioavailability based on AUC (invention, B/LIPANTHYL®67M): 532%.
Relative cₘₐₓ(invention, BA/LIPANTHYL®67M): 548%.
Relative bioavailability based on AUC (invention, C/LIPANTHYL®67M): 228%.
Relative cₘₐₓ(invention, C/LIPANTHYL®67M): 161%.
Relative bioavailability based on AUC (invention, D/LIPANTHYL®67M): 424%.
Relative cₘₐₓ(invention, D/LIPANTHYL®67M): 329%.

This invention may be embodied in other forms or carried out in other ways without departing from the spirit or essential characteristics thereof. The present disclosure is therefore to be considered as in all aspects illustrate and not restrictive, and all changes which come within the meaning and range of equivalency are intended to be embraced therein.

Various references are cited throughout this Specification, each of which is incorporated herein by reference in its entirety.

What is claimed is:

1. A particulate material comprising as an active substance fenofibrate and atorvastatin or a pharmaceutically active salt thereof, which provides a relative AUC₀₋₂₄ value (AUC_{fibric acid}/AUCₐₜₒᵣᵥₐₛₜₐₜᵢₙ) of between about 250 and about 10,000 when administered orally to a mammal, the AUC values being determined from steady state plasma concentrations of fibric acid and atorvastatin, respectively.

2. A particulate material according to claim 1, wherein the material provides a relative AUC₀₋₂₄ value of at least about 250, or at least about 500, or at least about 980, or at least about 2000.

3. A particulate material according to claim 1, wherein the material provides a relative AUC₀₋₂₄value of less than about 10,000, or less than about 5100, or less than about 4000, or less than about 2100.

4. A particulate material according to claim 1, wherein the material provides an AUC₀₋₂₄value of fibric acid (arithmetic mean) of at least 28,000 h·ng/L, or at least of about 40,000 h·ng/L, or at least of about 79,000 h·ng/L, or at least of about 118,000 h·ng/L.

5. A particulate material according to claim 1, wherein at least about 80% w/w of the total amount of active substances is dissolved in a vehicle selected from the group consisting of a hydrophobic, a hydrophilic and a water-miscible vehicles.

6. A particulate material according to claim 1, wherein at least about 85% w/w, at least about 90% w/w, at least about 95% w/w, at least about 98% w/w, at least about 99% w/w or at least about 99.9% w/w of the total amount of active substance is dissolved in the vehicle.

7. A particulate material according to claim 1 which is free-flowing.

8. A particulate material according to claim 1 further comprising one or more oil-sorption materials, which when tested as described herein
i) has an oil threshold value of about 10% or more, when tested according to the Threshold Test herein, and at least one of
ii) releases at least about 30% of an oil, when tested according to Release Test and
iii) in the form of a tablet has a disintegration time of at the most 1 hour, when tested according to Ph. Eur. Disintegration test, the tablet containing about 90% w/w or more of the oil-sorption material.

9. A particulate material according to claim 1, wherein the vehicle has a melting point of at the most about 250° C.

10. A particulate material according to claim 1, wherein the vehicle is hydrophobic.

11. A particulate material according to claim 6, wherein the hydrophobic vehicle is selected from the group consisting of straight chain saturated hydrocarbons, paraffins; fats and oils such as cacao butter, beef tallow, lard; low melting point waxes such yellow beeswax, white beeswax, carnauba wax, castor wax, japan wax, substituted and/or unsubstituted triglycerides, acrylic polymers, and mixtures thereof.

12. A particulate material according to claim 1, wherein the vehicle is hydrophilic or water-miscible.

13. A particulate material according to claim 12, wherein the hydrophilic or water-miscible vehicle is selected from the group consisting of polyethylene glycols, polyoxyethylene oxides, poloxamers, polyoxyethylene stearates, poly-epsilon caprolactone, fatty acids, monoglycerides, diglycerides, fatty alcohols, fractionated phospholipids and mixtures thereof.

14. A particulate material according to claim 12, wherein the hydrophilic or water-miscible vehicle is a polyglycolized glyceride.

15. A particulate material according to claim 12, wherein the hydrophilic or water-miscible vehicle is selected from the group consisting of polyvinylpyrrolidones, polyvinyl-polyvinylacetate copolymers (PVP-PVA), polyvinyl alcohol (PVA), polymethacrylic polymers, cellulose derivatives including hydroxypropyl methylcellulose (HPMC), hydroxypropyl cellulose (HPC), methylcellulose, sodium carboxymethylcellulose, hydroxyethyl cellulose, pectins, cyclodextrins, galactomannans, alginates, carragenates, xanthan gums, NVP polymers, PVP polymers and mixtures thereof.

16. A particulate material according to claim 13, wherein the vehicle is a polyethylene glycol (PEG).

17. A particulate material according to claim 16, wherein the polyethylene glycol has an average molecular weight of at least 1500.

18. A particulate material according to claim 12 comprising a mixture of two or more hydrophilic or water-miscible vehicles.

19. A particulate material according to claim 18, wherein the mixture comprises a polyethylene glycol and a poloxamer in a proportion of between about 1:3 and about 10:1, preferably between about 1:1 and about 5:1, more preferably between about 3:2 and about 4:1, especially between about 2:1 and about 3:1, in particular about 7:3.

20. A particulate material according to claim 19, wherein the poloxamer is poloxamer 188.

21. A particulate material according to claim 19, wherein the polyethylene glycol has an average molecular weight of about 6000 (PEG6000).

22. A particulate material according to claim 1, wherein the vehicle is non-aqueous.

23. A particulate material according to claim 1, wherein the concentration of the vehicle is at least about 10% w/w.

24. A particulate material according to claim 1, wherein the concentration of the vehicle is about 15% w/w or more, about 20% w/w or more, about 25% w/w or more, about 30% w/w or more, about 35% w/w or more or about 40% w/w or more.

25. A particulate material according to claim 1, wherein the fenofibrate is an analog thereof.

26. A particulate material according to claim 1, wherein the concentration of fenofibrate in the vehicle is at least about 10% w/w, based on the total weight of active substance and the vehicle.

27. A particulate material according to claim 1, wherein the concentration of fenofibrate in the vehicle is at least about 15% w/w, or at least about 16% w/w, or at least about 17% w/w, or at least about 20% w/w, preferably at least about 25% w/w, more preferably at least about 30% w/w, especially at least about 35% w/w, based on the total weight of active substance and the vehicle.

28. A particulate material according to claim 1, wherein the concentration of atorvastatin in the vehicle is at least about 1% w/w, based on the total weight of active substance and the vehicle.

29. A particulate material according to claim 1, wherein the concentration of atorvastatin in the vehicle is at least about 1.5% w/w, or at least about 2.5% w/w, or at least about 5% w/w, or at least about 7.5% w/w or at least about 10% w/w, based on the total weight of active substance and the vehicle.

30. A particulate material according to claim 1 having a moisture content of at the most about 2.5% w/w water.

31. A particulate material according to claim 1 having a moisture content of at the most about 2% w/w or 1% w/w water.

32. A particulate material according to claim 1 having a storage stability of about 2 months or more when tested at about 40° C. and about 75% RH.

33. A particulate material according to claim 1 having a storage stability of about 3 months or more, about 4 months or more, about 5 months or more or about 6 months or more when tested at about 40° C. and about 75% RH.

34. A particulate material according to claim 1, wherein the particulate material has a geometric weight mean diameter d_{gw} of ≧10 μm such as, e.g. ≧20 μm, from about 20 to about 2000, from about 30 to about 2000, from about 50 to about 2000, from about 60 to about 2000, from about 75 to about 2000 such as, e.g., from about 100 to about 1500 μm, from about 100 to about 1000 μm or from about 100 to about 700 μm, or at the most about 400 μm or at the most 300 μm such as, e.g., from about 50 to about 400 μm such as, e.g., from about 50 to about 350 μm, from about 50 to about 300 μm, from about 50 to about 250 μm or from about 100 to about 300 μm.

35. A particulate material according to claim 1, comprising one or more pharmaceutically acceptable excipients selected from the group consisting of fillers, disintegrants, binders, diluents, lubricants and glidants.

36. A particulate material according to claim 1, further comprising a pharmaceutically acceptable additive selected from the group consisting of flavoring agents, coloring agents, taste-masking agents, pH-adjusting agents, buffering agents, preservatives, stabilizing agents, anti-oxidants, wetting agents, humidity-adjusting agents, surface-active agents, suspending agents, absorption enhancing agents.

37. A particulate material according to claim 35, wherein at least one of the one or more pharmaceutically acceptable excipients are selected from the group consisting of silica acid or a derivative or salt thereof including silicates, silicon dioxide and polymers thereof; magnesium aluminosilicate and/or magnesium aluminometasilicate, bentonite, kaolin, magnesium trisilicate, montmorillonite and/or saponite.

38. A particulate material according to claim 37 comprising a silica acid or a derivative or salt thereof.

39. A particulate material according to claim 37 comprising silicon dioxide or a polymer thereof.

40. A particulate material according to claim 37 comprising AEROPERL® 300.

41. A solid dosage form comprising a particulate material as defined in claim 1.

42. A solid dosage form according to claim 41 having a storage stability of about 2 months or more when tested at about 40° C. and about 75% RH.

43. A solid dosage form according to claim 41 having a storage stability of about 3 months or more, about 4 months or more, about 5 months or more or about 6 months or more when tested at about 40° C. and about 75% RH.

44. A dosage form according to claim 41, wherein at least about 75% of the fenofibrate and/or the atorvastatin is released from the composition within about 45 min when tested in an in vitro dissolution test according to Ph. Eur. dissolution test (paddle) employing water with about 0.75% sodium lauryl sulfate as dissolution medium, about 50 rpm and a temperature of about 37° C.

45. A solid dosage form according to claim 44, wherein the dissolution test is carried out after about 1 month of storage at a temperature of about 40° C. and a relative humidity of about 75%.

46. A solid dosage form according to claim 41, wherein the concentration of the particulate material is in a range of from about 5% to 100% w/w such as, e.g., from about 10% to about 90% w/w, from about 15% to about 85% w/w, from about 20% to about 80% w/w, from about 25% to about 80% w/w, from about 30% to about 80% w/w, from about 35% to about 80% w/w, from about 40% to about 75% w/w, from about 45% to about 75% w/w or from about 50% to about 70% w/w of the dosage form.

47. A solid dosage form according to claim 46, wherein the concentration of the particulate material is about 50% w/w or more of the dosage form.

48. A solid dosage form according to claim 41 comprising a multiplicity of individual units such as, e.g., pellets, beads and/or granules.

49. A solid dosage form according to claim 41 in the form of tablets, capsules or sachets.

50. A solid dosage form according to claim 41 in the form of a tablet.

51. A solid dosage form according to claim 50, wherein the tablet is coated with a coating selected from the group consisting of film coatings, modified release coatings, enteric coatings, protective coatings and anti-adhesive coatings.

52. A solid dosage form according to claim 41, wherein the active substances are embedded in a matrix that releases the fenofibrate by diffusion.

53. A solid dosage form according to claim 52, wherein the matrix remains substantially intact during the period of drug release.

54. A solid dosage form according to claim 41, wherein the active substances are embedded in a matrix that release the fenofibrate by eroding.

55. A solid dosage form according to claim 41, wherein the active substances are released from the dosage form by diffusion through a substantially water-insoluble coating.

56. A solid dosage form according to claim 41 in the form of a polydepot dosage form, which—upon administration—disintegrates into a multiplicity of individual units from which the active substances are released.

57. A solid dosage form according to claim 41 having a moisture content of at the most about 2.5% w/w water.

58. A solid dosage form according to claim 41 having a moisture content of at the most about 2% w/w or 1% w/w water.

59. A solid dosage form according to claim 41 in unit dosage form, wherein the unit dosage form comprises from about 130 to about 170 mg of fenofibrate and from about 5 to about 80 mg of atorvastatin or a pharmaceutically acceptable salt thereof.

60. A solid dosage form according to claim 41 in unit dosage form, wherein the unit dosage form comprises about 160 mg of fenofibrate, or about 145 mg of fenofibrate or of a pharmaceutically acceptable salt thereof.

61. A solid dosage form according to claim 41 in unit dosage form, wherein the unit dosage form comprises about 10 mg of atorvastatin, or about 15 mg of atorvastatin, or about 20 mg of atorvastatin, or about 30 mg of atorvastatin, or about 40 mg of atorvastatin, or of a pharmaceutically acceptable salt of atorvastatin.

62. A solid dosage form according to claim 41 in unit dosage form, wherein the unit dosage form comprises fenofibrate and atorvastatin or pharmaceutically acceptable salt thereof and the weight ratio between fenofibrate and atorvastatin or a pharmaceutically acceptable salt thereof is from about 2:1 to about 40:1.

63. A solid dosage form according to claim 41, which results in an increased bioavailability of fenofibrate relative to existing commercial fenofibrate dosage forms when administered to a mammal in need thereof.

64. A solid dosage form according to claim 41, wherein the fenofibrate and/or the atorvastatin or a pharmaceutically acceptable salt thereof is stable.

65. A solid dosage form according to claim 64, wherein the fenofibrate and/or the atorvastatin or a pharmaceutically acceptable salt thereof is present in an amount of at least 90%, or at least 95%, or at least 100%, relative to the amount prior to storage, when assayed after 3 months of storage at a temperature of about 40° C. and a relative humidity of about 75%.

66. A method of manufacturing the solid oral dosage form of claim 41 comprising the steps of:
i) Bringing a vehicle in liquid form, if applicable,
ii) Maintaining the liquid vehicle of (i) at a temperature below the melting point of the fenofibrate and/or the atorvastatin or a pharmaceutically acceptable salt thereof,
iii) Dissolving the desired amount of fibrate and atorvastatin in the vehicle of (ii) to obtain a solution,
iv) Spraying the resulting solution of (iii) onto a solid carrier having a temperature below the melting point of the vehicle to obtain a composition,
v) Mechanically working the resulting composition of (iv) to obtain particles, i.e. a particulate material, and
vi) Optionally subjecting the particulate material to conventional methods for preparing solid dosage forms.

67. A method of manufacturing the solid oral dosage form of claim 41 comprising the steps of:
a) obtaining a particulate material comprising fenofibrate comprising:
i) Bringing a vehicle in liquid form, to obtain a liquid vehicle,
ii) Maintaining the liquid vehicle of i) at a temperature below the melting point of fenofibrate or a pharmaceutically acceptable salt thereof,
iii) Dissolving the desired amount of fenofibrate in the vehicle of ii) to obtain a solution,
iv) Spraying the resulting solution of iii) onto a solid carrier having a temperature below the melting point of the vehicle to obtain a composition,
v) Mechanically working the resulting composition of iv) to obtain particles, i.e. a particulate material containing fenofibrate,
b) obtaining a particulate material containing atorvastatin comprising the steps of;
i) Bringing a vehicle in liquid form to obtain a liquid vehicle,
ii) Maintaining the liquid vehicle of i) at a temperature below the melting point of atorvastatin or a pharmaceutically acceptable salt thereof,
iii) Dissolving the desired amount of atorvastatin in the vehicle of ii) to obtain solution,
iv) Spraying the resulting solution of iii) onto a solid carrier having a temperature below the melting point of the vehicle to obtain a composition,
v) Mechanically working the resulting composition of iv) to obtain particles, i.e. a particulate material containing atorvastatin, followed by the steps of
c) Mixing the particulate material containing fenofibrate and the particulate material containing atorvastatin, and
d) Optionally subjecting the particulate material to conventional methods for preparing solid dosage forms.

68. The method according to claim 67, wherein a particulate material containing atorvastatin of step b) is obtained prior to obtaining a particulate material containing fenofibrate.

69. The method according to claim 67, wherein a particulate material containing atorvastatin of step b) is obtained simultaneously with obtaining a particulate material containing fenofibrate.

70. The method according to claim 67, wherein a particulate material containing atorvastatin of step b) is obtained after obtaining a particulate material containing fenofibrate.

71. A method of manufacturing the solid oral dosage form of claim 41 comprising the steps of:
a) obtaining a particulate material comprising fenofibrate comprising:
i) Bringing vehicle in liquid form to obtain a liquid vehicle,
ii) Maintaining the liquid vehicle of i) at a temperature below the melting point of fenofibrate or a pharmaceutically acceptable salt thereof,
iii) Dissolving the desired amount of fenofibrate in the vehicle of ii) to obtain a solution,
iv) Spraying the resulting solution of iii) onto a solid carrier having a temperature below the melting point of the vehicle to obtain composition,
v) Mechanically working the resulting composition of iv) to obtain particles, i.e. a particulate material containing fenofibrate,
b) Micronizing atorvastatin or a pharmaceutically acceptable salt thereof, if applicable, followed by the steps of
c) Mixing the particulate material containing fenofibrate and micronized atorvastatin, and
d) Optionally subjecting the particulate material to conventional methods for preparing solid dosage forms.
